(19) United States
(12) Patent Application Publication
(10) Pub. No.: US 2024/0074668 A1
(43) Pub. Date: Mar. 7, 2024

(54) SYSTEMS AND METHODS FOR DETERMINATION OF PULSE ARRIVAL TIME WITH WEARABLE ELECTRONIC DEVICES

(71) Applicant: Board of Regents of the University of Nebraska, Lincoln, NE (US)

(72) Inventors: Cody Anderson, Blair, NE (US); Song-Young Park, Elkhorn, NE (US)

(21) Appl. No.: 18/240,481

(22) Filed: Aug. 31, 2023

Related U.S. Application Data
(60) Provisional application No. 63/403,497, filed on Sep. 2, 2022.

(51) Int. Cl. A61B 5/021; A61B 5/00; A61B 5/352

(52) U.S. Cl. CPC A61B 5/02125; A61B 5/0059; A61B 5/352; A61B 5/7221; A61B 5/7225; A61B 5/7232; A61B 5/726

(57) ABSTRACT

Systems and methods for determining pulse arrival time utilizing sensors coupled to mobile electronic devices are described. A system embodiment includes, but is not limited to, a sensor configured to provide electrocardiogram (ECG) data; an optical sensor configured to provide optical data; and a controller configured to access each of the ECG data and the optical data, the controller configured to: isolate and normalize R-wave information from the ECG data, isolate information associated with the cardiac rhythm from the isolated and normalized R-wave information to provide pulse waves, determine temporal characteristics of the pulse waves, convert and normalize the optical data in a wavelet time-frequency plane, and calculate pulse arrival time utilizing each of the temporal characteristics of the pulse waves and the converted and normalized optical data.

### CROSS-REFERENCE TO RELATED APPLICATIONS

The present application claims the benefit under 35 U.S.C. § 119(e) of U.S. Provisional Application Ser. No. 63/403,497, filed Sep. 2, 2022, and titled “SYSTEMS AND METHODS FOR DETERMINATION OF PULSE ARRIVAL TIME WITH WEARABLE ELECTRONIC DEVICES,” which is herein incorporated by reference in its entirety.

### BACKGROUND

Blood pressure is an attribute that healthcare professionals recommend measuring and tracking for various health diagnoses and can be considered one of the vital signs of a healthcare patient. Blood pressure relates to the force of blood pushing against the walls of arteries as the heart pumps blood through the vascular system. High blood pressure, or hypertension, can result in health complications such as cardiovascular disease, stroke, dementia, eye conditions, and renal conditions. Low blood pressure, or hypotension, can cause light headedness, vision issues, fatigue, fainting, and shock. Blood pressure and vascular stiffness can affect pulse arrival time, the time taken by pulse waves to propagate between two points in a patient's vasculature.

### SUMMARY

Systems and methods for stable and noise-resistant determination of pulse arrival time (PAT) using signals from electrocardiogram sensors (ECGs) and optical sensors are described. Methods described herein provide flexibility regarding signal amplitudes of output from optical sensors and stability against noise for relatively low signal-to-noise ratio sensors provided on wearable electronic devices while permitting determination of a reliable quality index of the PAT determinations.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The patent or application file contains at least one drawing executed in color. Copies of this patent or patent application publication with color drawing(s) will be provided by the Office upon request and payment of the necessary fee.

The Detailed Description is described with reference to the accompanying figures. In the figures, the use of the same reference numbers in different instances in the description and the figures may indicate similar or identical items.

FIG. 1A is an environmental view of a system for determination of pulse arrival time using sensor data originating from a wearable electronic device, in accordance with example embodiments of the present disclosure.

FIG. 1B is an environmental view of a system for determination of pulse arrival time using sensor data originating from a wearable electronic device, in accordance with example embodiments of the present disclosure.

FIG. 2 is a method for determination of pulse arrival time using sensor data originating from a wearable electronic device, in accordance with example embodiments of the present disclosure.

FIG. 3 is an example diagram of an ECG data set showing amplitudes over time.

FIG. 4 is a method for removing background data and normalizing R-wave information from the ECG data, in accordance with example embodiments of the present disclosure.

FIG. 5 is an example diagram of an ECG data set illustrating an application of a high pass filter.

FIG. 6 is an example diagram of an ECG data set illustrating a zeroing process of negative amplitude values.

FIG. 7 is an example diagram of an ECG data set illustrating amplification of the positive filtered ECG data.

FIG. 8 is an example diagram of a Mortlet wavelet used in wavelet transformation.

FIG. 9 is an example diagram of an amplified ECG data set following wavelet transformation.

FIG. 10 is an example diagram of an ECG data set in the wavelet time-frequency plane illustrating reduction to a one-dimensional data set.

FIG. 11 is an example diagram of a one-dimensional ECG data set illustrating establishment of a binary ECG vector containing temporal locations of R-waves.

FIG. 12 is an overlay diagram of the binary ECG vector of FIG. 11 and the original ECG signal.

FIG. 13 is an example diagram of wavelet transformation of the binary ECG vector of FIG. 11.

FIG. 14 is a method for isolating information associated with the cardiac rhythm from the wavelet transformation of the binary ECG vector to obtain pulse waves, in accordance with example embodiments of the present disclosure.

FIG. 15 is an example diagram of the R-R interval overlayed on the wavelet transformation of the binary ECG vector of FIG. 13.

FIG. 16 is an example diagram of heart rate over time overlayed on the wavelet transformation of the binary ECG vector of FIG. 13.

FIG. 17 is an example diagram of the ECG data having isolated amplitudes from the wavelet time-frequency plane within a confidence interval.

FIG. 18 is a method for determining temporal characteristics of pulse waves, in accordance with example embodiments of the present disclosure.

FIG. 19 is a real component wavelet time-frequency plane based on the real components of the wavelet coefficients of the binary ECG vector.

FIG. 20A is an example diagram illustrating elementwise multiplication of a matrix of real components from the ECG signal with a normalized amplitude matrix from the ECG signal.

FIG. 20B is an example diagram illustrating elementwise multiplication of a matrix of real components from the ECG signal with a normalized amplitude matrix from the ECG signal.

FIG. 21 is an example diagram of an original ECG signal and a resultant ECG signal following isolation and normalization of the R-wave information from the original ECG signal.

FIG. 22A is an example diagram of optical data for use with the wavelet transformation.

FIG. 22B is an example diagram of a wavelet transformation of the optical data of FIG. 22A with amplitude normalization.

FIG. 23 is a method for calculating PAT based on wavelet time-frequency planes of ECG signal data and optical signal data, in accordance with example embodiments of the present disclosure.

FIG. 24A is an example diagram illustrating trimming time borders of the wavelet time-frequency planes of the ECG and optical signals with alignment for elementwise product-sum derivation.

FIG. 24B is an example diagram illustrating results of elementwise product-sum derivation of the aligned and trimmed wavelet time-frequency planes of the ECG and optical signals.

FIG. 25 is an example diagram illustrating determination of product-sums for all time-shift iterations and determination of a local maximum representative of an average PAT of pulse waves.

FIG. 26 is an example diagram illustrating phase angle differences between phase angles of isolated ECG coefficients and optical coefficients.

FIG. 27 is an example diagram illustrating determination of a synchronization index timeseries with multi-resolution synchronization that reduces a two-dimensional synchronization index matrix to a one-dimensional timeseries that assigns one synchronization index to each point in time.

FIG. 28 is an example diagram illustrating elementwise multiplication of the synchronization time series across the frequency dimension of each of an ECG and NIRS 2-D complex time-scale planes.

FIG. 29A is an example diagram illustrating experimental results of correlation coefficient values for a plurality of signal-to-noise ratios.

FIG. 29B is an example diagram illustrating experimental results of product-sum values for a plurality of signal-to-noise ratios.

FIG. 29C is an example diagram illustrating experimental results of synchronization index values for a plurality of signal-to-noise ratios.

FIG. 29D is an example diagram illustrating experimental results of absolute error values for a plurality of signal-to-noise ratios.

FIG. 30A is an example diagram illustrating experimental results of synchronization index values for a plurality of test subjects.

FIG. 30B is an example diagram illustrating experimental results of number of synchronization index values for the group of test subjects from FIG. 30A.

FIG. 30C is an example diagram illustrating experimental results of pulse arrival time values for a plurality of test subjects.

FIG. 30D is an example diagram illustrating experimental results of number of pulse arrival time values for the group of test subjects from FIG. 30C.

FIG. 31A is an example diagram illustrating experimental results of synchronization index as a function of simulated errors with and without the use of multi-resolution synchronization for signals obtained from a plurality of test subjects.

FIG. 31B is an example diagram illustrating absolute error in PAT as a function of simulated errors with and without the use of multi-resolution synchronization for the signals obtained from a plurality of test subjects from FIG. 31A.

FIG. 32A is an example diagram illustrating experimental results of the relationship between PAT and diastolic blood pressure from one test subject using multi-resolution synchronization.

FIG. 32B is an example diagram illustrating experimental results of the relationship between PAT and mean blood pressure from one test subject using multi-resolution synchronization.

FIG. 32C is an example diagram illustrating experimental results of the relationship between PAT and systolic blood pressure from one test subject using multi-resolution synchronization.

### DETAILED DESCRIPTION

### Overview

Portable fitness trackers and other wearable electronic devices have become widely available to the public and provide personalized and on-demand health monitoring, which may aid in the prevention and treatment of cardiovascular diseases. Modern fitness trackers are outfitted with various sensors that provide electrocardiogram (ECG) and photoplethysmography (PPG) outputs. With these sensors, information can be extracted relating to heart rate, autonomic nervous system activity, and heart electrical activity. While these data are useful, they do not provide a complete assessment of cardiovascular health. For example, blood pressure is an important and tightly regulated cardiovascular variable, and the dysregulation of blood pressure is a dangerous and common facet of many cardiovascular diseases.

However, the sensors utilized by wearable electronic devices are susceptible to instability by relying on low signal-to-noise ratio sensor outputs and are insufficient to recreate traditional conditions used for blood pressure determination directly. For instance, a traditional method for determining blood pressure can involve inflating and deflating a cuff wrapped about a limb to restrict and subsequently release blood flow through a blood vessel and noting the systolic blood pressure and the diastolic blood pressure via auscultation, or sound-based recognition of blood flow. Systolic blood pressure is associated with the indication of blood turbulence in the vessel (e.g., Korotkoff sounds) and diastolic blood pressure is associated with the cessation of the blood flow sounds. The sensors of wearable electronic devices are unsuitable for recreating the restriction and release of blood flow to analyze the characteristic sounds or conditions attributable to systolic and diastolic blood pressure.

Other methods to analyze characteristics associated with blood pressure and vascular stiffness rely on determination of pulse arrival time (PAT). PAT can refer to the time it takes for pressure waves generated by the heart to propagate through the vascular system and can be used to assess blood pressure. Example methods to acquire pulse signals include tonometry and ultrasonography techniques applied to large conduit arteries of a patient to provide high-fidelity output signals. Geometry-based methods in the time domain (e.g., intersecting tangents methods) can be used to determine the temporal location for the derivation of PAT based on the high-fidelity output signals from the tonometry and ultrasonography techniques. However, low signal-to-noise ratio sensor outputs, such as those provided by electrocardiogram (ECG) and photoplethysmography (PPG), or other sensor outputs on wearable electronic devices are prone to lower signal-to-noise ratio than conduit artery signals, thereby drastically reducing the reliability of geometry-based methods to derive PAT. Additionally, output signals from sensors integrated into wearable electronic devices include high amounts of irrelevant information that confounds detection of information relevant for the determination of PAT, which introduces instability into PAT calculations. Additionally, traditional methods for signal processing, such as hard-thresholding (e.g., setting a constant voltage value as the binary threshold to determine if an event exists in a signal), can introduce error for systems employed across a wide population (e.g., consumer-grade fitness trackers), were the signal amplitudes of data from integrated sensors varies from person to person.

Accordingly, the present disclosure is directed, at least in part, to systems and methods for stable and noise-resistant determination of PAT using signals from sensors integrated into wearable electronic devices. Example sensors and outputs include ECG and optical signals, such as PPG and near-infrared spectroscopy (NIRS). Methods described herein provide flexibility regarding signal amplitudes of output from optical sensors and stability against noise for relatively low signal-to-noise ratio sensors while permitting determination of a reliable quality index of the PAT determinations. For example, raw ECG and optical signals can be converted to different data forms for reliably deriving PAT while eliminating hard-thresholding. In implementations, the signal conversion includes a stepwise procedure of filtering and normalizing signal information and incorporating continuous wavelet transform and pattern recognition techniques to determine an average PAT based on ECG and optical data signals. Quality indexes can include one or more of a synchronization index and a multi-resolution synchronization index, which in turn can be utilized to derive PAT.

### Example Implementations

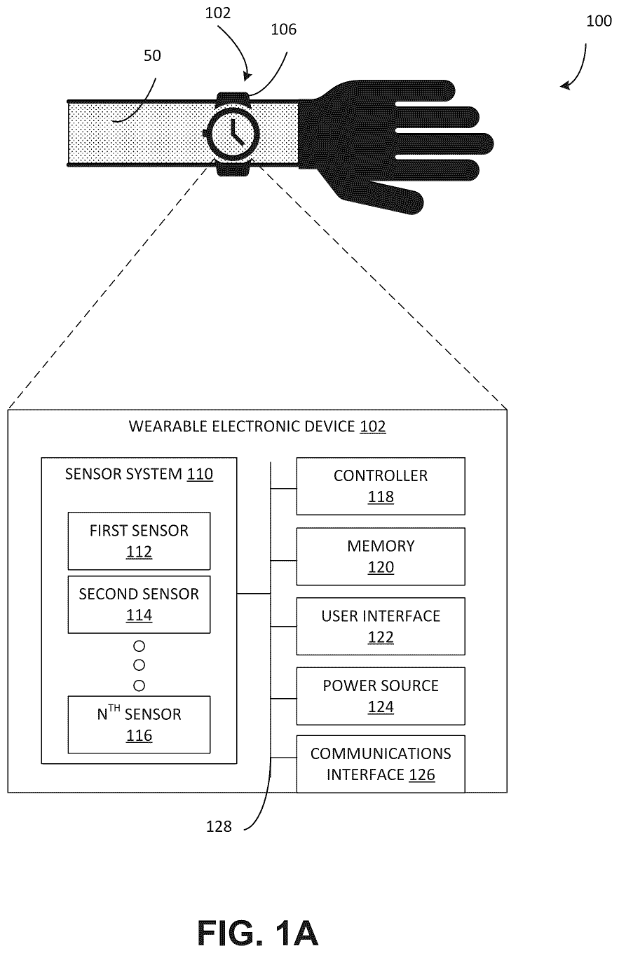

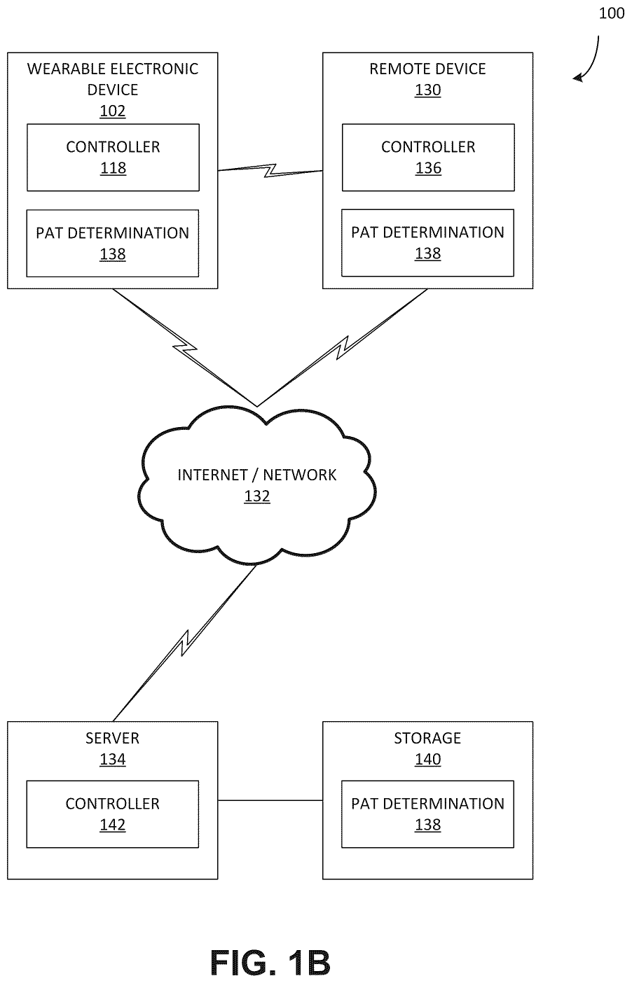

Referring to FIGS. 1A and 1B, a system 100 for determination of PAT is described. The system 100 is shown generally including a wearable electronic device 102 having a sensor system 104 configured to measure health metrics of a user on which the wearable electronic device 102 is positioned. For example, the wearable electronic device 102 is shown configured as a wearable electronic watch or fitness tracker positioned on a user's wrist 50 secured by a wristband 106, however the system 100 is not limited to such configuration and can include additional or alternative arrangements of the wearable electronic device 102 and the user including, but not limited to, positioning the wearable electronic device 102 on the user's torso, phalange, appendage, neck, head, or the like with corresponding attachment devices (e.g., clip, strap, adhesive, etc.). The wearable electronic device 102 includes a housing 108 to structurally support the components of the wearable electronic device 102. For instance, components can be mounted externally to the housing 108, internally within the housing 108, or combinations thereof to support the components on the user during wear.

The wearable electronic device 102 includes a sensor system 110 configured to measure characteristics of the user, characteristics of the environment in which the user is located, characteristics of one or features or components of the wearable electronic device 102, or combinations thereof. For example, the sensor system 110 can include a plurality of sensors including a first sensor 112, a second sensor 114, and additional sensor(s) 116. In implementations, the first sensor 112 includes a sensor configured to output ECG data (e.g., an “ECG sensor”) and the second sensor 114 includes an optical sensor. The optical sensor can include, but is not limited to, a PPG sensor, a NIRS sensor, or combinations thereof. The output signals from the sensor system 110 are used by the system 100 to determine PAT information for the user, which can be processed directly by the wearable electronic device 102, through intermediary communication/processing devices, or combinations thereof, as described herein.

The wearable electronic device 102 can include additional components to facilitate operation of the wearable electronic device 102. For example, the wearable electronic device 102 is shown including a controller 118, a memory 120, a user interface 122, a power source 124, and a communications interface 126 coupled to a bus 128. The controller 118 functions with other components of the wearable electronic device 102, such as to execute functionality of applications stored in the memory 120, to direct storage to and transfer from data in the memory 120, to manage functionality of the wearable electronic device 102, or the like. In implementations, the controller 118 includes one or more computer processors that can work independently, as a group or subgroup, in combination with other logic components on the wearable electronic device 102 or remote from the wearable electronic device 102, or combinations thereof, to determine PAT of the user based on signal output from the sensor system 110. The memory 120 can include, but is not limited to, a random access memory (RAM), a read-only memory (ROM), a flash memory, a redundant array of disks (RAID), a hard disk, a network attached storage (NAS), an optical disc, a non-optical disc or other storage media, or combinations thereof. In implementations, the memory 120 stores instructions for execution by the controller 118 to determine PAT of the user based on signal output from the sensor system 110, such as by implementing method 200, described herein.

The user interface 122 is configured to receive user interaction with one or more features of the wearable electronic device 102 (e.g., via integrated button(s), touch screen, microphone, etc.), to output information to the user (e.g., via display screen, audio output, vibrational output, etc.), or combinations thereof. In implementations, the user interface 122 is utilized by the user to initiate operation of ECG and optical sensors of the sensor system 110 to provide data used by the system 100 to determine PAT of the user. The power source 124 can be any suitable power supply to power the wearable electronic device 102, such as a battery, solar cell, kinetic energy storage, or the like. The communications interface 126 facilitates data transfer to and from the wearable electronic device 102. For example, the communications interface 126 can be configured for wireless data transfer, wired data transfer, or combinations thereof. Wireless data transfer can occur, for example, over radio signal, Wi-Fi signal, Bluetooth signal, cellular communication signal, or combinations thereof.

The wearable electronic device 102 can communicate with one or more remote systems via the communications interface 126 for the determination of PAT of the user. Referring to FIG. 1B, the system 100 is shown with the wearable electronic device 102 in communication with a remote device 130, an internet/network 132, and a server 134 to facilitate data transfer, distributed processing of system functionality, and the like. The remote device 130 can include, but is not limited to, a mobile communications device (e.g., a smartphone), a tablet computer, a mobile computer, a laptop computer, a desktop computer, or another computing/communications device configured to communicate with the wearable electronic device 102. In implementations, the remote device 130 includes a local controller 136 configured to facilitate calculation of PAT of the user via processing of instructions, software, code, logic, or the like stored locally on the remote device 130 (e.g., shown as 138) based on sensor signals received from the wearable electronic device 102 (e.g., signals from the sensor system 110). In implementations, the remote device 130 accesses the PAT calculation functionality from the server 134 via the internet/network 132. For example, the server 134 can host or otherwise access the PAT calculation functionality in a storage device 140 for communication or transfer to one or more of the remote device 130 or the wearable electronic device 102. In implementations, the server 134 includes a controller 142 configured to facilitate calculation of PAT of the user via processing of instructions, software, code, logic, or the like stored locally on the server 134 or otherwise accessible to the server 134 (e.g., shown as 138). The system 100 supports local computing of PAT (e.g., via the wearable electronic device 102) and distributed computing of PAT (e.g., via one or more remote devices in combination with sensor signals from the wearable electronic device 102).

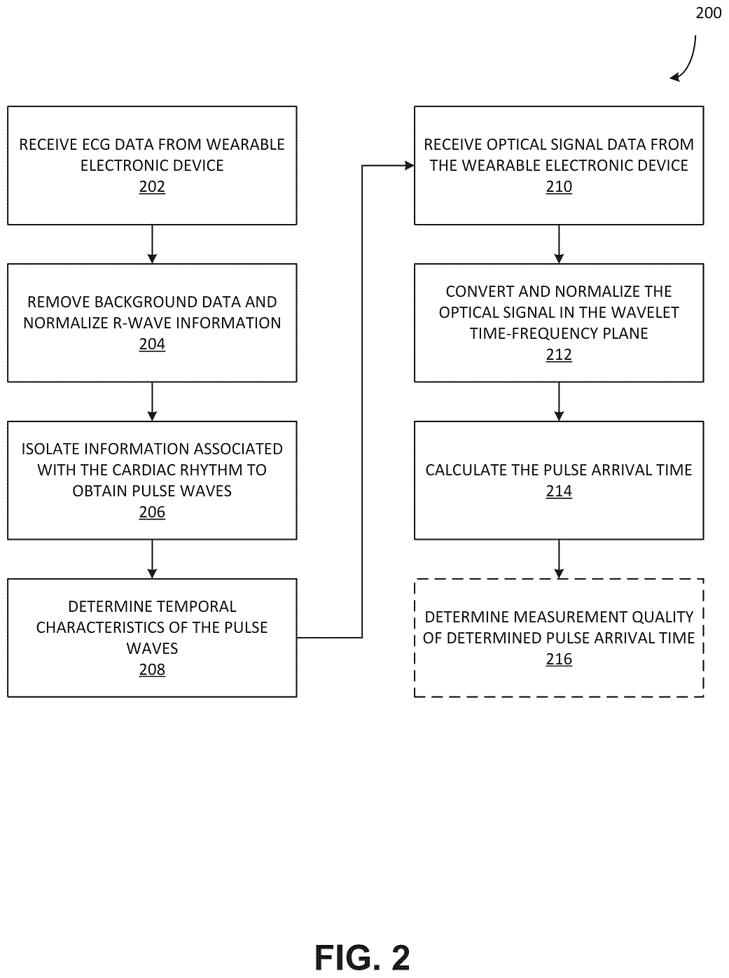

Referring to FIG. 2, a method 200 for determining PAT using signals from electrocardiogram sensors and optical sensors is described. The method 200 is shown including steps for receiving ECG data from a wearable electronic device (in block 202), removing background data and normalizing R-wave information from the ECG data (in block 204), isolating information associated with cardiac rhythm to obtain pulse waves (in block 206), determining temporal characteristics of the pulse waves (in block 208), receiving optical signal data from the wearable electronic device (in block 210), converting and normalizing the optical signal in the wavelet time-frequency plane (in block 212), calculating PAT (in block 214), and determining a measurement quality of the determined PAT (in block 216). Each of these steps is described in further detail herein.

Method 200 includes receiving ECG data from a wearable electronic device (in block 202). The ECG data can be sourced from an ECG sensor as part of the sensor system 110 on the wearable electronic device 102, from a separate system in remote communication with the wearable electronic device 102, or combinations thereof. In implementations, the ECG data is collected by forming a lead to orient the direction of the electrical dipole of the ECG, where lead I is produced from the electrodes interfacing with the user and the lead is completed by having the user touch the wearable electronic device 102 with the contralateral hand. Examples shown herein correspond to a 30-second sampling period, however the disclosure is not limited to such sampling period. For example, the sampling period could be less than 30 second or more than 30 seconds without departing from the scope of the disclosure.

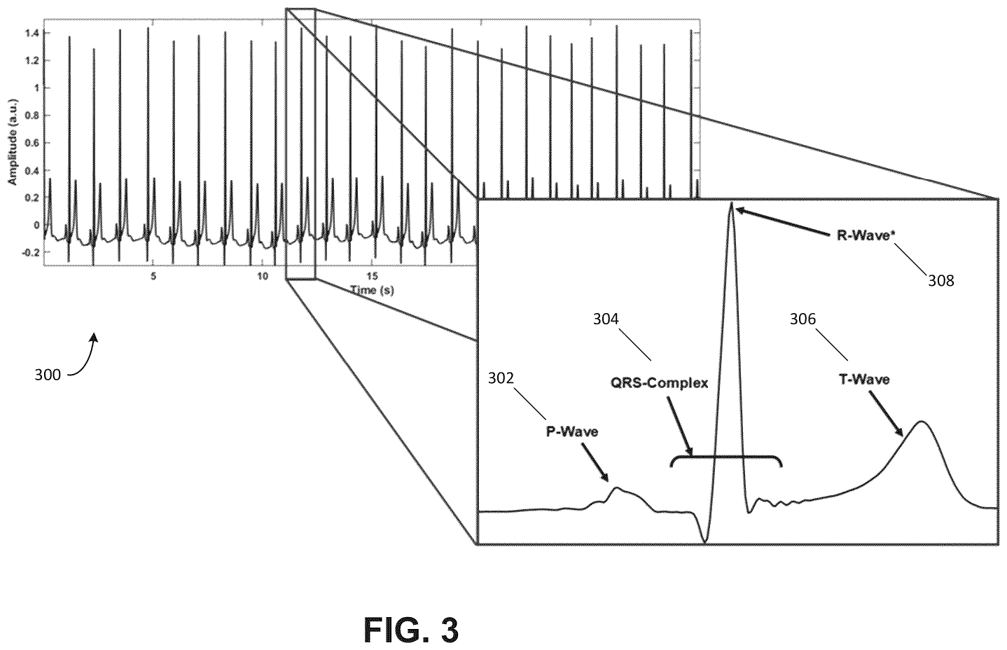

The ECG data can include multiple features including, but not limited to a P-wave, a QRS complex, and a T-wave. For example, FIG. 3 illustrates an example ECG data set 300 with amplitudes over time, where the P-wave is shown as reference character 302 and includes an initial peak, the QRS-complex is shown as reference character 304 and includes an initial downward deflection following the P-wave, a sharp upward deflection following the initial downward deflection, and a downward deflection or series of smaller peaks following the upward deflection, and the T-wave is shown as reference character 306 and includes an upward deflection following the QRS-complex. The QRS complex includes the R-wave, shown as reference character 308, which can facilitate the derivation of PAT because the R-wave is a highly time-localized and high amplitude event that indicates ventricular myocardial depolarization and the beginning of ventricular systole. The R-wave is unique among the features of the ECG data because it is frequently the highest frequency and highest amplitude event. The R-wave can be utilized as a reliable indicator of when the pulse wave is generated with each cardiac cycle. In implementations, method 200 utilizes the R-wave as the beginning of pulse wave generation in the derivation of PAT.

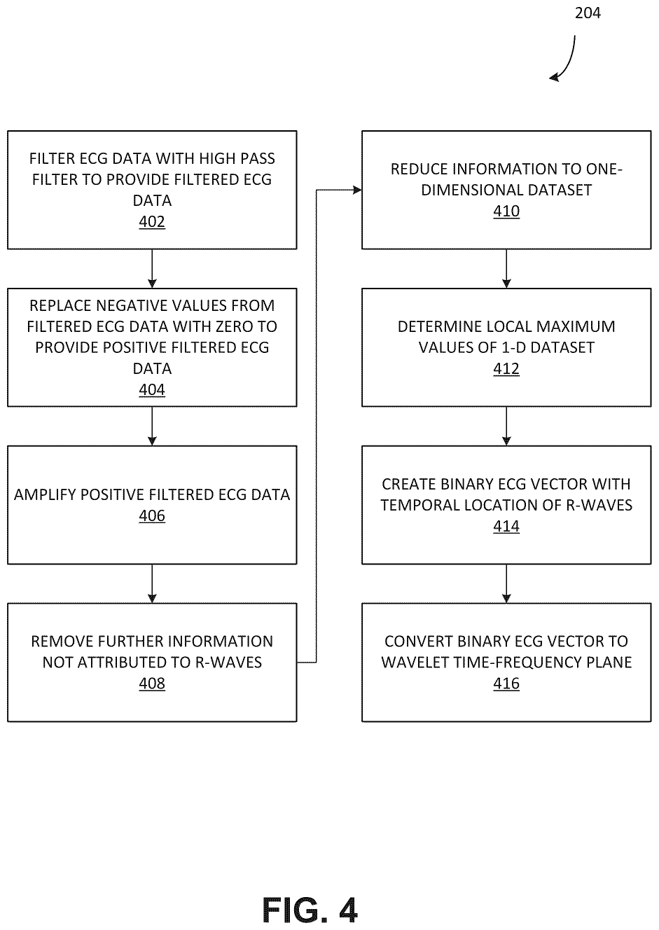

Method 200 includes removing background data and normalizing R-wave information from the ECG data (in block 204). While the ECG data includes the R-wave, the ECG data can also include oscillations that are irrelevant to the cardiac related R-wave (e.g., other portions of the QRS complex, the P-wave, the T-wave, etc.). Method 200 can include sequential and amplitude-independent techniques to eliminate irrelevant information (e.g., amplitudes of low frequency information) from the ECG signal to reliably isolate the location of R-waves. Referring to FIG. 4, an example of removing background data and normalizing R-wave information from the ECG data (block 204) is shown. For instance, removing background data and normalizing R-wave information from the ECG data generally includes filtering the ECG data with a high pass filter to provide filtered ECG data (in block 402), replacing negative values from the filtered ECG data with zero to provide positive filtered ECG data (in block 404), amplifying the positive filtered ECG data (in block 406), removing further information not attributed to R-waves from the positive filtered ECG data (in block 408), reducing the information to a one-dimensional dataset (in block 410), determining local maximum values in the one-dimensional dataset (in block 412), creating a binary ECG vector containing temporal locations of R-waves (in block 414), and converting the binary ECG vector to the wavelet time-frequency plane (in block 416). Each of these steps is described in further detail herein.

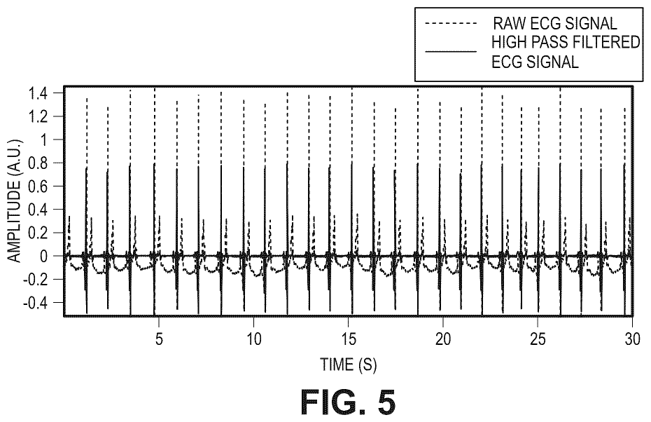

In block 402, the ECG data received from the wearable electronic device 102 is filtered with a high pass filter. The high pass filter facilitates isolating the R-waves from the ECG signal. For instance, high pass filtering the signal removes low frequency oscillations and trends, centers the signal about zero, and accentuates the amplitude of the R-wave relative to the amplitudes of irrelevant low-frequency features in the signal since the R-wave is the highest frequency event of the other features of the ECG signal (e.g., P-wave, QRS complex, T-wave). In implementations, an example of which is shown in FIG. 5, the high pass filter includes a moving average window of length 1/20 seconds. In FIG. 5, the high pass filter attenuates the R-wave amplitude less than the other features of the ECG trace (P-wave, T-wave, etc.) since the R-wave spectrum contains more energy in the high frequencies relative to the other features in the ECG data. Additionally, the low frequency trends of the ECG trace have been removed, and the signal is centered about zero. While method 200 describes using a high pass filter, the disclosure is not limited to such filtering techniques and can include other filtering techniques in addition to or as an alternative to high pass filtering.

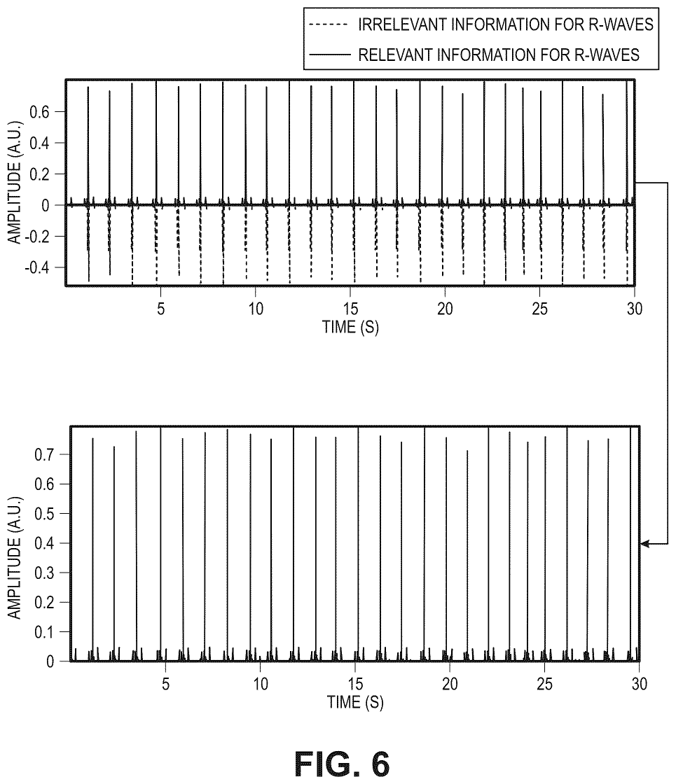

After the high pass filter, the ECG signal will be centered about zero, with information contained both above and below zero. Because of the orientation of the ECG sensor lead, the R-wave is positive, so information below zero is irrelevant for determining the temporal location of the R-wave. The method block 204 includes replacing negative values from the filtered ECG data with zero to provide positive filtered ECG data (in block 404), which can aid with amplification of the signal. An example of the transition from the filtered ECG data to the positive filtered ECG data is shown in FIG. 6, where negative amplitudes of the filtered ECG data are removed.

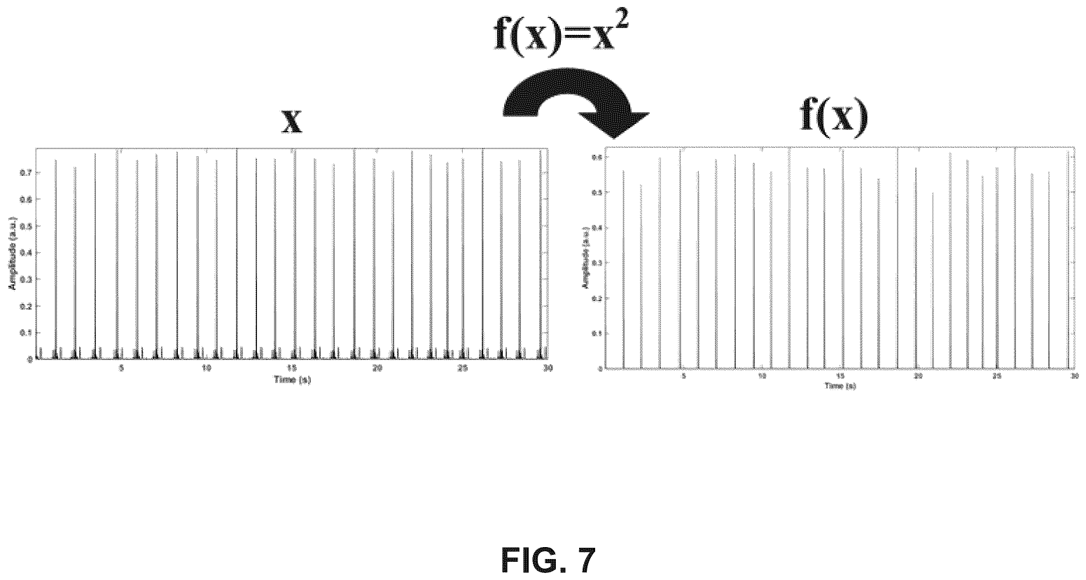

The method block 204 includes amplifying the positive filtered ECG data (in block 406). In the positive filtered ECG signal, the R-waves will be the highest amplitude events in the series. Because of this feature, if multiplicative or exponential operations are repeatedly applied to the series, the amplitudes of the R-waves will increase to infinity faster or decay to zero slower than the amplitudes of the other features in the signal. Amplification of the signal can therefore disproportionally elevate the R-wave amplitude above the amplitudes of the other events in the series to assist with isolating and normalizing the R-wave. In implementations, an example of which is shown in FIG. 7, the amplification involves squaring each element in the positive filtered ECG data series. While the example shows amplification by squaring, the disclosure is not limited to such amplification and can include additional or alternative amplification factors, where such factors can be modified depending on the relative amplitudes of the R-waves to other features in the signal. In implementations, the greater the amplification factor, the more prominently the R-waves will be detected as compared to other artifacts in the signal.

The method block 204 includes removing further information not attributed to R-waves from the positive filtered ECG data (in block 408). For instance, the method 200 can apply a wavelet transform to the amplified ECG data. In implementations, the wavelet transform can include a continuous wavelet transform:

c(s,t)=∫_{-∞}^{∞}Ψ_{s,t}^{*}(u)g(u)du (Eq. 1)

where Ψ_{s,t}(u) is a family of wavelets derived from the mother wavelet dilated to the ‘s’ scale, and translated in time, ‘t,’ g(u) is the one-dimensional time-series of interest, c(s, t) is the two-dimensional time-scale matrix of complex coefficients that contain the spectral properties (i.e., spectral amplitudes and phase angles) of the original signal, g(u), and ‘*’ represents the complex conjugate.

Dilation and translation of the mother wavelet generates a family of wavelets defined as:

$${Ψ}_{s , t} = {\vert s \vert}^{- p} ψ ( \frac{u - t}{s} ) \qquad ( Eq . 2 )$$

where Ψ_{s,t} represents the family of wavelets derived from the mother wavelet

$$ψ ( \frac{u - t}{s} )$$

by dilating with scale, ‘s’, and time-shifting by time, ‘t’, and is normalized by the factor |s|^{-p}, where ‘s’ is the scale for dilating the mother wavelet, and ‘p’ is an arbitrary factor, where small values of ‘p’ weight the spectral amplitudes towards lower frequencies, and large values of ‘p’ weight the spectral amplitudes towards higher frequencies.

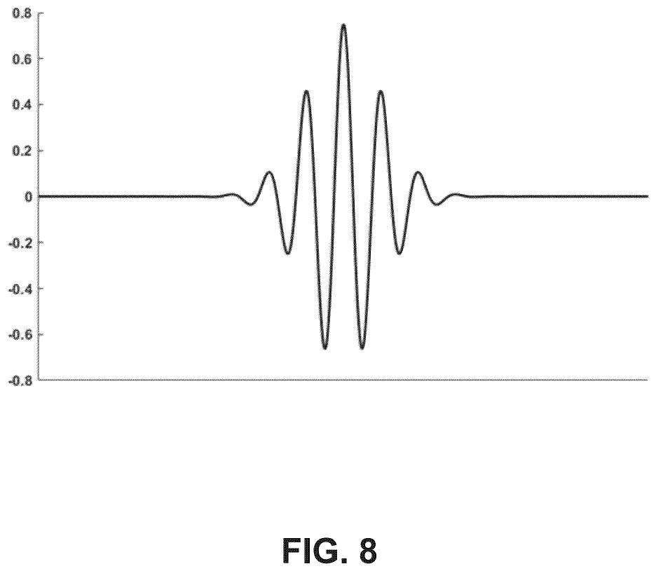

In implementations, the method 200 utilizes a Morlet wavelet as the mother wavelet for the wavelet transform procedure, an example of which is shown in FIG. 8. The Morlet wavelet is defined as:

$$ψ ( u ) = \frac{1}{\sqrt[4]{π}} {e}^{- i {ω}_{o} u} {e}^{- {u}^{2} / 2} \qquad ( Eq . 3 )$$

where ψ(u) represents the Morlet mother wavelet, which is modulated by a complex sinusoid, e^{-iω}^{ₒ}^{u}, with central frequency, ωₒ, and a gaussian function,

$$\frac{1}{\sqrt[4]{π}} {e}^{- {u}^{2} / 2} .$$

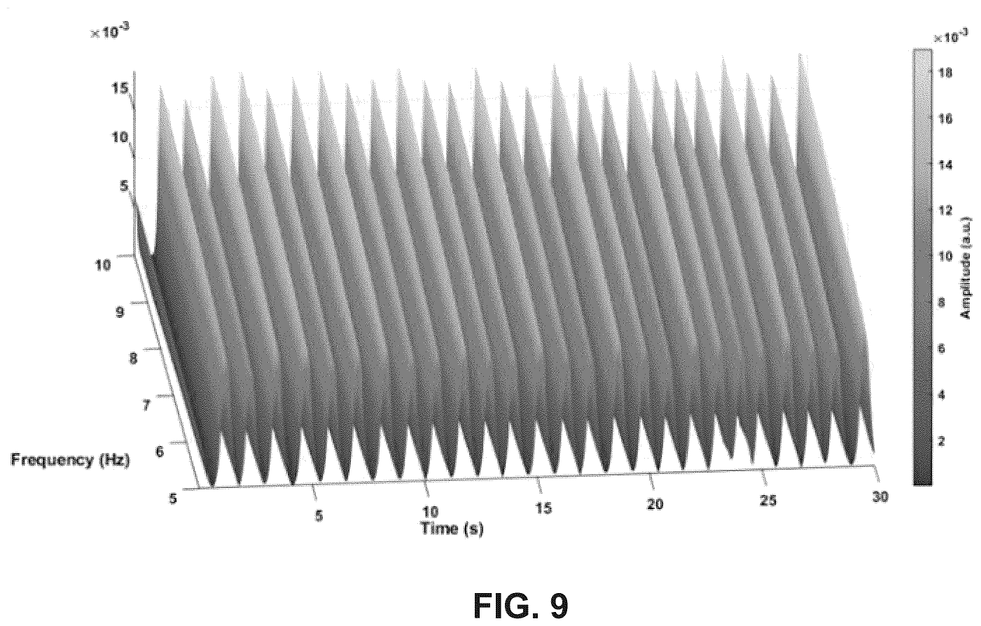

Referring to FIG. 9, an example wavelet transformation of the amplified ECG data with the Morlet wavelet as the mother wavelet with a central frequency (ωₒ) of 2π and with the transform applied for linearly partitioned scales between the frequencies of about 5 to about 10 Hz. The Morlet wavelet can be dilated to alter its frequency. Lower frequency wavelets are larger and therefore have reduced time resolution, whereas higher frequency wavelets are small and highly time localized, thereby improving time resolution. The trade-off in overall resolution is between the time and frequency domains. While low frequency wavelets have poor time resolution, they have precise frequency resolution. Likewise, while high frequency wavelets have precise time resolution, high frequency wavelets are not well localized in frequency. Since the wavelets can have resolution trade-offs, wavelets can be used to blur the information in the amplified ECG time series, which further eliminates information unrelated to the R-waves. After amplification, the amplitude of the R-waves greatly exceeds that of the other features of the signal, so the coefficients of the wavelet transform are heavily weighted to provide information about the R-waves. The wavelet transform within the frequency band of about 5 to about 10 Hz produces high amplitude bands in the time-frequency plane that align with the location of R-waves, with higher frequency wavelets providing higher temporal precision regarding the location of the R-waves than lower frequency wavelets. While the example shows a frequency band of about 5 to about 10 Hz, the disclosure is not limited to such frequency band and can include additional or alternative frequencies, where such frequencies can be modified depending on features in the signal.

As described, greater amplification of the positive high pass filtered ECG signal can provide more accurate identification of the R-waves, particularly where the coefficients in the wavelet transform are weighted more heavily towards the R-waves. Motion artifacts can produce high frequency and high amplitude artifacts in the ECG signal that may be equal to or larger than the amplitude of the R-waves. In this case, the information associated with the motion artifact can be present in block 408 of the method 200. However, even if the amplitudes of motion artifacts are greater than the R-waves, and the amplification process amplifies the motion artifacts to a greater extent than the R-waves, this will not impact the detection of the R-waves because the wavelets are time-localized. As such, a motion artifact can potentially influence time adjacent R-waves, but would not jeopardize the integrity of the entire signal. Further, the method 200 can include a measurement quality of the ultimately derived PAT (in block 216) to account for potential motion artifacts.

The method block 204 includes reducing the information to a one-dimensional dataset (in block 410). In implementations, the information in the ECG signal can be reduced to a simple sinusoid with maximums that correspond to the location of the R-waves. For example, once the wavelet transform has been applied to the amplified ECG signal (e.g., in block 408), the pseudo-integral of the wavelet amplitudes across the frequency bandwidth for each partition in time is calculated as the sum of the respective wavelet amplitudes. This reduces the wavelet coefficients to a one-dimensional time series that resembles a simple sinusoidal wave, an example of which is shown in FIG. 10. As shown in FIG. 10, the minimums of the sinusoid are more dilated than the maximums of the sinusoid. This is because the lower frequency wavelets have lower time resolution and blur the information between R-waves to a greater extent than the high frequency wavelets. Because of this, the amplitudes associated with the high frequency wavelets provide most of the information associated with the maximums in the sinusoid, and the lower frequency wavelet provides more information associated with the minimums in the sinusoid. The use of wavelets across a bandwidth of frequencies balances both temporal precision and R-wave detection accuracy because the high frequency wavelets provide exceptional temporal precision and the low frequency wavelets eliminate information about signal amplitudes between R-waves, thereby reducing the probability of producing maximums that are not associated with the R-waves.

The method block 204 includes determining local maximum values in the one-dimensional dataset (in block 412). In implementations, the first and second derivative rules are applied to the one-dimensional dataset to determine the location of local maximums throughout the signal, where the local maximums are taken to represent the location of R-waves.

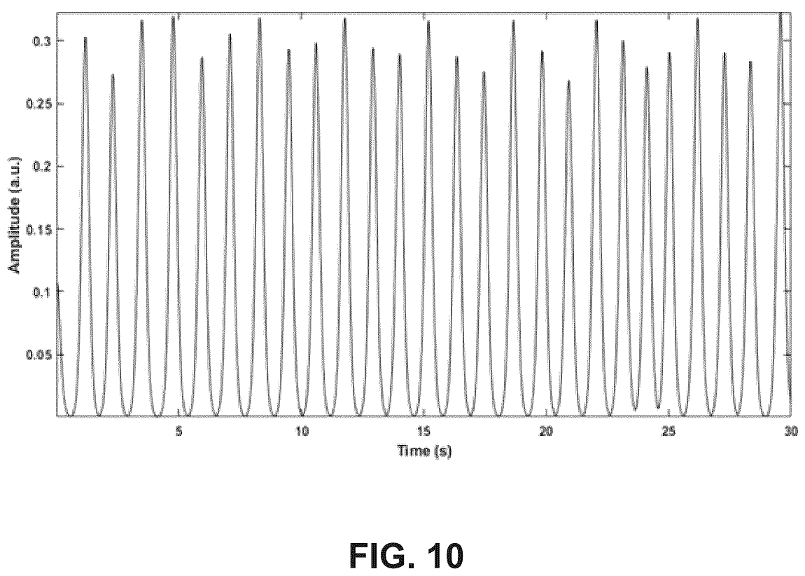

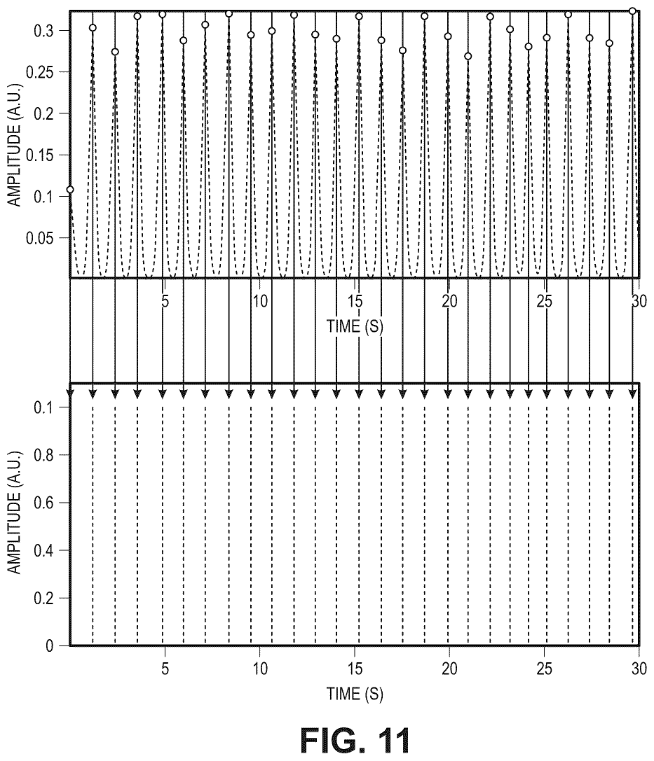

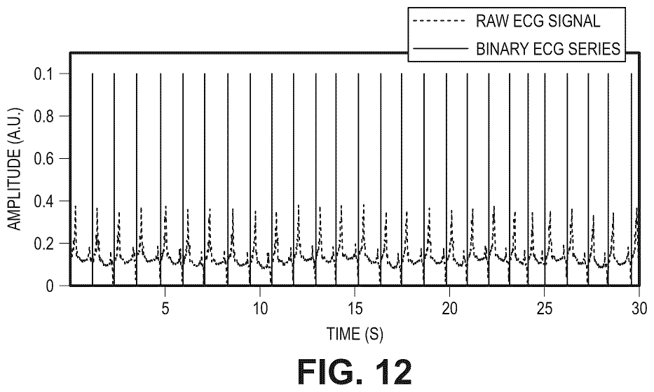

The method block 204 includes creating a binary ECG vector containing temporal locations of R-waves (in block 414). In implementations, an example of which is shown in FIG. 11, a vector of zeros is produced that is the same length as the original ECG signal and the locations in time where an R-wave is present are replaced with the value of one to store the information about the temporal locations of R-waves. Creating the binary ECG vector reduces the information in the original ECG signal to an amplitude normalized vector that contains the information about the time-location of the R-waves. For example, FIG. 12 shows the original ECG signal overlaid with the binary ECG vector, which illustrates precise agreement between locations of the R-waves in each of the original ECG signal and the binary ECG vector. The method 200 can detect the R-waves independent of the original amplitude of the R-waves in the original ECG signal. As such, the method 200 is applicable for detecting R-waves of various users of the wearable electronic device 102 when the R-wave amplitudes cannot be assumed to be constant within or between users.

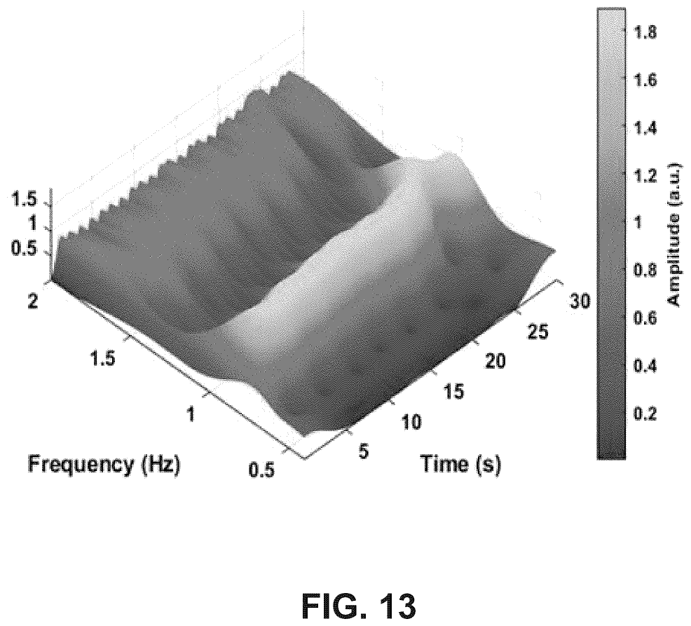

The method block 204 includes converting the binary ECG vector to the wavelet time-frequency plane (in block 416). In implementations, the wavelet transform is applied to the binary ECG vector using a bandwidth of frequencies from about 0.4 to about 2 Hz to capture heart rates within traditional resting ranges while accounting for upper and lower buffering. An example conversion is shown in FIG. 13, where wavelet transform is applied with the Morlet wavelet as the mother wavelet and ωₒ equal to 2π and with the scaling factor in the wavelet transform (|s|^{-p}) is removed from the procedure. Removal of the scaling factor reduces the amplitudes of lower heart rates relative to higher heart rates, since a significant portion of the binary ECG vector is composed of zeros. Because the scaling parameter is removed, the amplitudes of the wavelet coefficients can be interpreted as how well the R-waves align with the peaks of a wavelet of a given dilation (i.e., how well the R-waves align with a given frequency). The coefficient amplitudes can be interpreted as the likelihood of a given frequency existing in the signal at a given point in time. Higher amplitudes represent frequencies that have a higher probability of existing than lower amplitudes. While the example shows a bandwidth of frequencies from about 0.4 to about 2 Hz, the disclosure is not limited to such bandwidth and can include additional or alternative frequencies, where such frequencies can be modified depending on features in the signal.

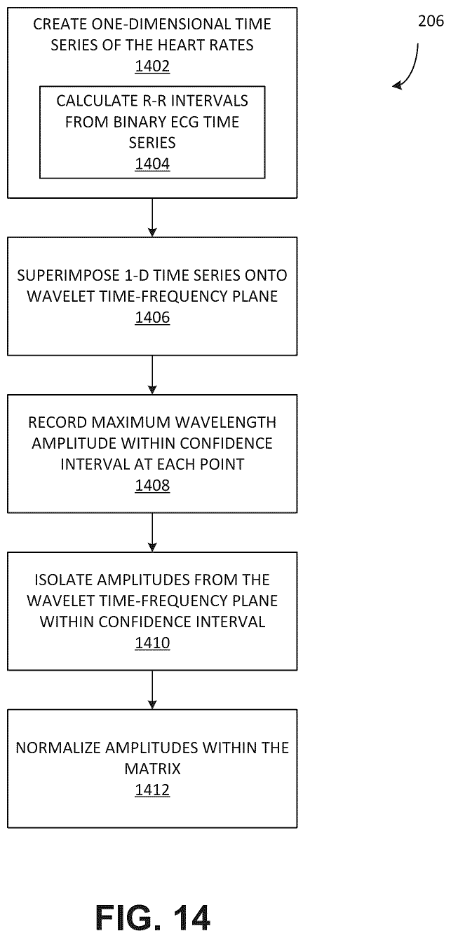

Following transformation of the ECG signal to the wavelet time-frequency plane in method block 204, the method 200 proceeds to block 206 where information associated with the cardiac rhythm is isolated, removing portions of the signal unrelated to the cardiac rhythm. Referring to FIG. 14, an example of isolating information associated with the cardiac rhythm to obtain pulse waves (block 206) is shown. For instance, isolating information associated with the cardiac rhythm to obtain pulse waves generally includes creating a one-dimensional time series of heart rates associated with the ECG signal (in block 1402), superimposing the one-dimensional time series onto the wavelet time-frequency plane (in block 1406), recording the maximum wavelength amplitude within a confidence interval at each point (in block 1408), isolating amplitudes from the wavelet time-frequency plane within the confidence interval (in block 1410), and normalizing amplitudes within the matrix (in block 1412). Each of these steps is described in further detail herein.

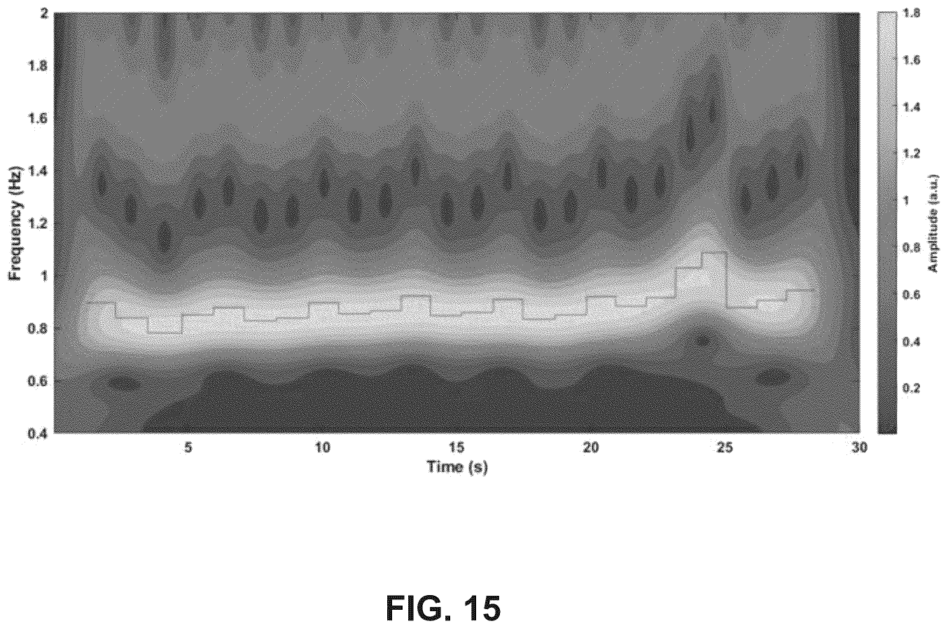

In block 1402, a one-dimensional time series of the heart rates is created utilizing the binary ECG time series from block 204. In implementations, the one-dimensional time series is provided by calculating the R-R interval (e.g., intervals from R-wave to R-wave) from the binary ECG time series from block 204 (in block 1404). The method block 206 also includes superimposing the one-dimensional time series onto the wavelet time-frequency plane (in block 1406). An example is shown in FIG. 15, where the R-R interval heart rate is overlayed in red over the time-frequency plane and demonstrates strong agreement with the coefficient amplitudes derived with the wavelet transform. Since the R-R interval derived heart rate and the wavelet transform observe the same information to derive the cardiac cadence, the R-R interval heart rate demonstrates a strong coupling to the band of amplitudes associated with the heart rate in the wavelet transformation.

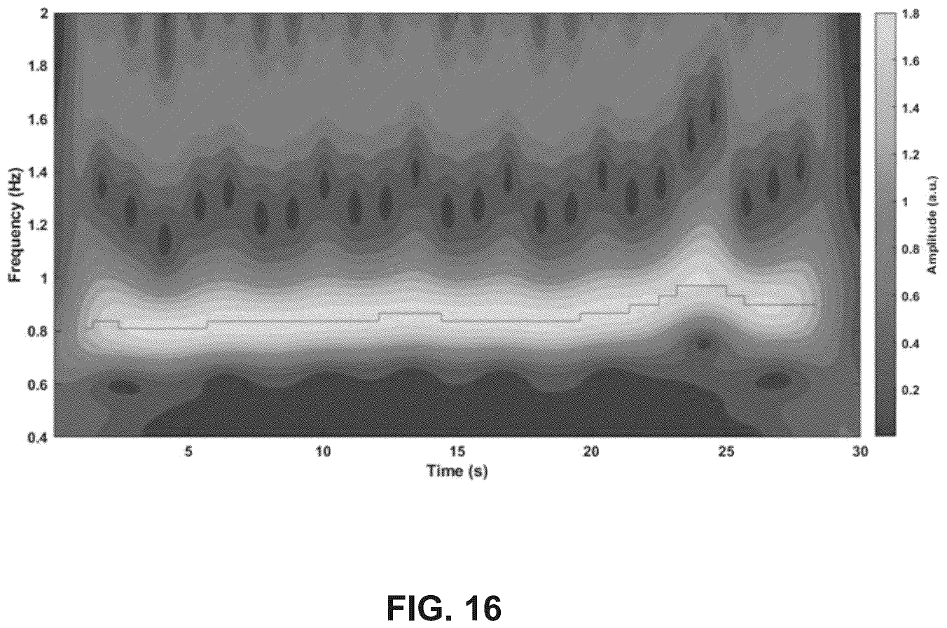

The method block 206 includes recording the maximum wavelength amplitude within a confidence interval at each point (in block 1408). The wavelet transform provides imprecise correlation between the time-domain and the frequency-domain and may not obtain perfect time and frequency resolution with the wavelet transform (e.g., due to Heisenberg's Principle of Uncertainty). As such, the energy associated with a given frequency in a signal is smeared across adjacent frequencies and is not perfectly frequency localized. This means that information about the cardiac rhythm is not simply localized to the maximum amplitude in the wavelet time-frequency plane, but information related to the cardiac rhythm exists in frequencies adjacent to the maximum amplitudes. The distribution of signal energy across a slice in the wavelet time-frequency plane for a time series with a sinusoid of a pure frequency is a gaussian distribution when the frequency domain is expressed in scales (period of the frequency), and the relationship between the central scale (maximum amplitude of the gaussian) and the standard deviation of the gaussian is the central scale divided by 2π when ωₒ of the Morlet wavelet is 2π. In implementations, 95% confidence intervals are created about the R-R interval derived heart rates across time, and the maximum wavelet coefficient amplitude within each 95% confidence interval is recorded at each point in time. This creates a time series that maps the heart rate across time as derived with the wavelet transform, an example of which is shown in FIG. 16. While the example shows a 95% confidence interval, the disclosure is not limited to such confidence interval and can include additional or alternative confidence intervals dependent on features in the signal.

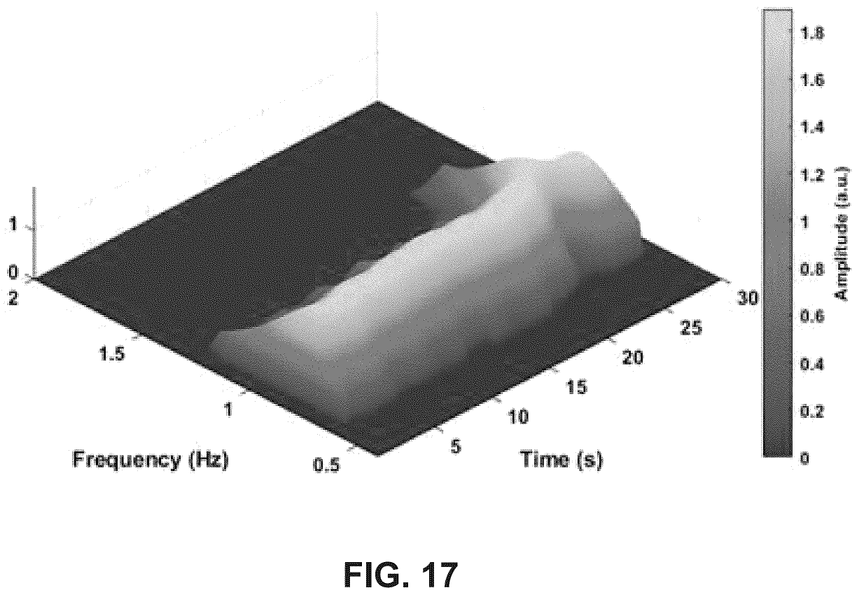

The method block 206 includes isolating amplitudes from the wavelet time-frequency plane within the confidence interval (in block 1410). In implementations, the wavelet derived heart rate time series is superimposed onto the wavelet time-frequency plane and 95% confidence intervals are produced about the wavelet derived heart rate frequencies, where amplitudes contained within the 95% confidence intervals are maintained and data outside the 95% confidence intervals can be discarded (e.g., reduced to zero). An example of isolating amplitudes from the wavelet time-frequency plane within the confidence interval is shown in FIG. 17, which shows that the coefficient amplitudes can be three-dimensional in nature. The method block 206 further includes normalizing amplitudes within the matrix (in block 1412). In implementations, amplitudes within the matrix are normalized as a ratio of the maximum amplitude in the matrix to provide isolated and normalized amplitude information related to the cardiac rhythm, however the amplitudes may not directly provide information about the temporal characteristics of the pulse waves.

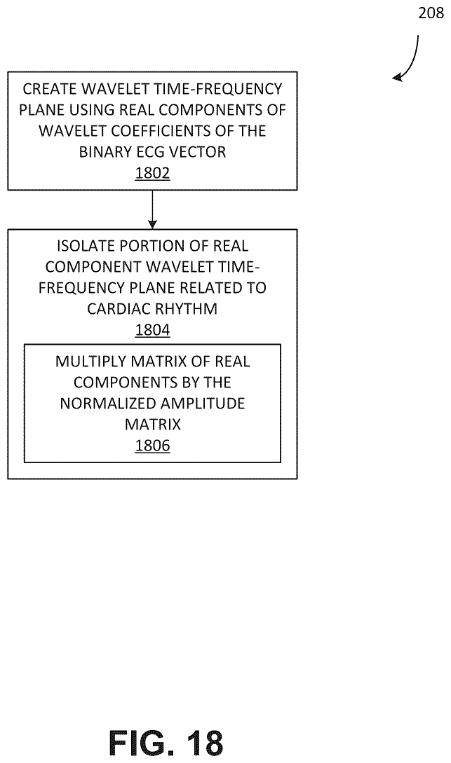

Method 200 includes determining temporal characteristics of the pulse waves (in block 208). Referring to FIG. 18, an example of determining temporal characteristics of the pulse waves (block 208) is shown. For instance, determining temporal characteristics of the pulse waves generally includes creating a wavelet time-frequency plane using the real components of the wavelet coefficients of the binary ECG vector (in block 1802) and isolating the portion of the real component wavelet time-frequency plane related to cardiac rhythm (in block 1804). Each of these steps is described in further detail herein.

In block 1802, a wavelet time-frequency plane is created using the real components of the wavelet coefficients of the binary ECG vector (e.g., from block 416). The real components of the coefficients represent the pure sinusoidal components of the signal at each point in time for each frequency. An example real component wavelet time-frequency plane is shown in FIG. 19. Complex wavelet transformations produce complex coefficients with real and imaginary components. The real components of the coefficients represent the pure sinusoidal components of the signal at a given frequency. The real components of the coefficients therefore provide timing information about the wave-trains in the signal, which are not contained in the amplitudes of the coefficients. The real component wavelet time-frequency plane shown in FIG. 19 highlights agreement with the wavelet time-frequency plane shown in FIG. 13 (which is produced from the amplitudes of the coefficients), in part due to the amplitude being a function of both the real and imaginary components of the complex coefficients.

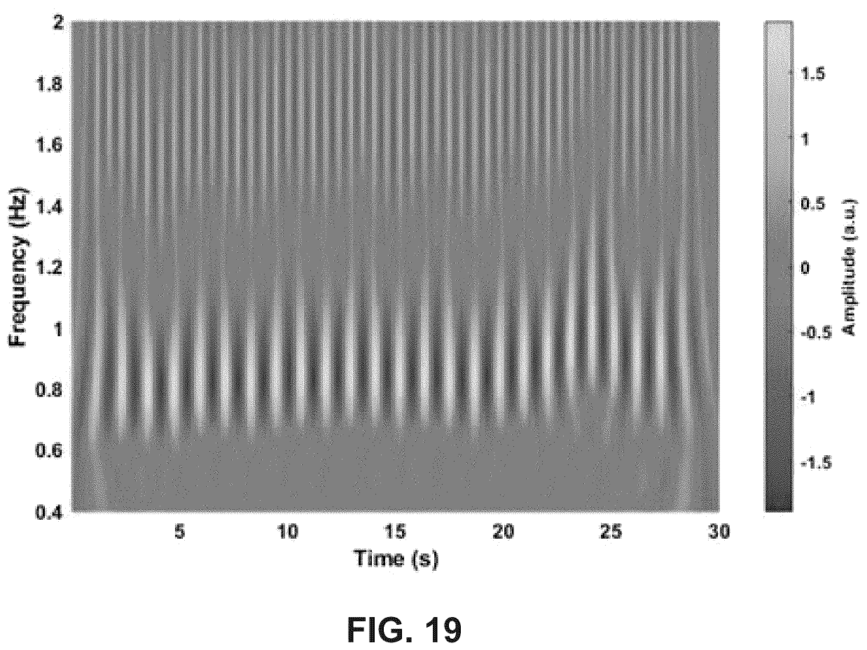

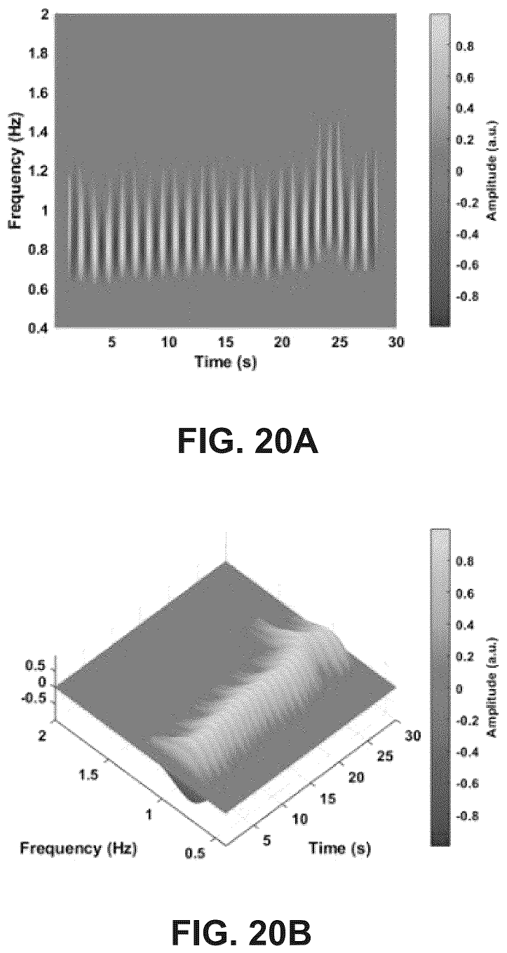

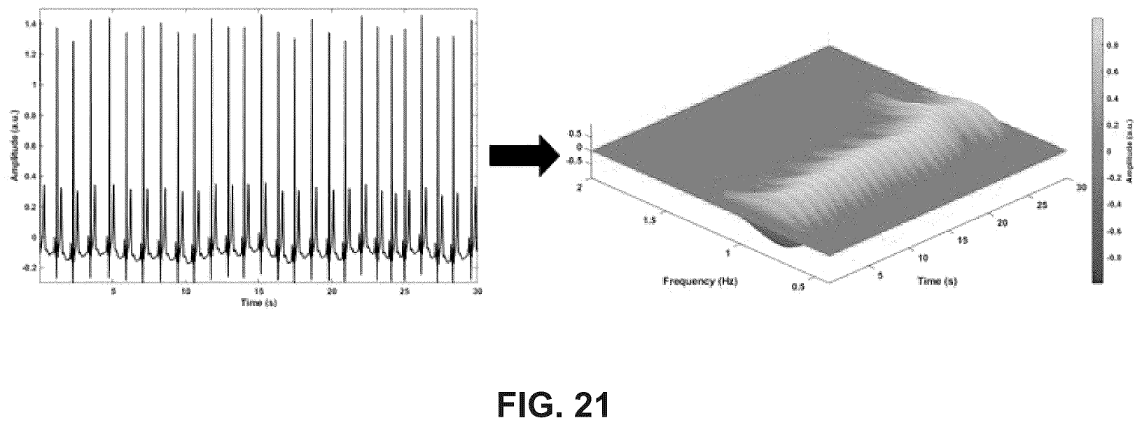

The method block 208 also includes isolating the portion of the real component wavelet time-frequency plane related to cardiac rhythm (in block 1804). In implementations, the band of sinusoids related to the cardiac rhythm is isolated by multiplying the matrix of real coefficients (e.g., from block 1802) by the normalized amplitude matrix (e.g., from block 1412) in an elementwise fashion (in block 1806). Examples are shown in FIGS. 20A and 20B, where sinusoids not associated with the heart rate band are removed and the frequencies within the heart rate band are weighted according to the highest probability frequencies. The resultant ECG signal is a three-dimensional sinusoidal wave-train in the wavelet time-frequency plane that is free to vary with the sinus arrythmia across time. A comparison of the original ECG signal and the resultant ECG signal from method 200 is shown in FIG. 21.

The method 200 also includes receiving optical signal data from the wearable electronic device (in block 210). The optical signal from the wearable electronic device 102 can be used as a comparator for the resultant ECG signal. For instance, the optical signal can be less reliable for isolating information about the cardiac rhythm as compared to the ECG signal, so a converted and normalized form of the optical signal is used to compare against the resultant ECG signal in the wavelet time-frequency plane. In implementations, the optical signal data is provided from one or more of a photoplethysmography (PPG) sensor, a near-infrared spectroscopy (NIRS) sensor, or another optical sensor. The optical signal data can be retrieved directly from the optical sensor, through an intermediate data transfer system, retrieved from a computer memory (e.g., located on the wearable electronic device 102, located on a remote server, located on a mobile communication device, or the like), or combinations thereof.

The method 200 also includes converting and normalizing the optical signal in the wavelet time-frequency plane (in block 212). In implementations, the optical signal is transformed from the time domain to the wavelet time-frequency plane with the Morlet wavelet as the mother wavelet with ωₒ equal to 2π and uses a bandwidth of frequencies from about 0.4 to about 2 Hz to capture heart rates within traditional resting ranges while accounting for upper and lower buffering. The bandwidth of frequencies can be the same or similar to the bandwidth of frequencies used in the treatment of the ECG data. As with the ECG signal, the frequencies used for the example of optical signal treatment can be modified and are not limited to those described herein.

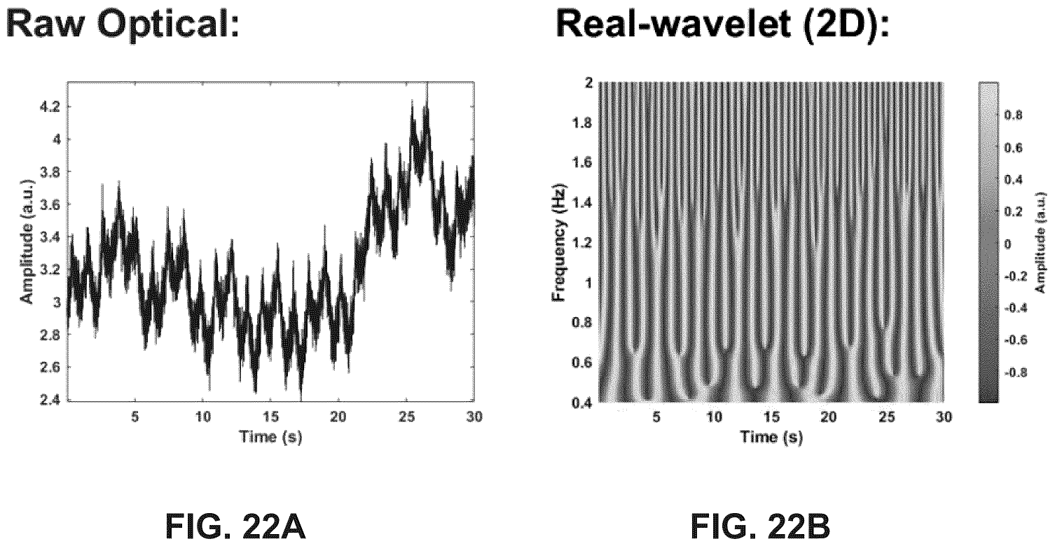

Referring to FIG. 22B, an example wavelet transformation of the optical data (e.g., shown in FIG. 22A) is shown. In the example, phase angles of the optical signal coefficients are used as the basis to normalize the optical signal instead of amplitudes of the optical signal (e.g., since the optical signal amplitudes can be viewed as less reliable for providing information about the cardiac rhythm). For instance, the phase angles can be derived from the wavelet coefficients (ranging from 0 to 2π) and the cosine of these phase angles can be used to reconstruct the real components of the coefficients. Since cosine ranges from −1 to 1, the oscillations in the wavelet time-frequency plane are normalized between the values of −1 to 1. As a result, the oscillations in the optical signal can be equally weighted in block 212, where the weighing of the heart rate band can be attributed to the ECG signal during the determination of PAT.

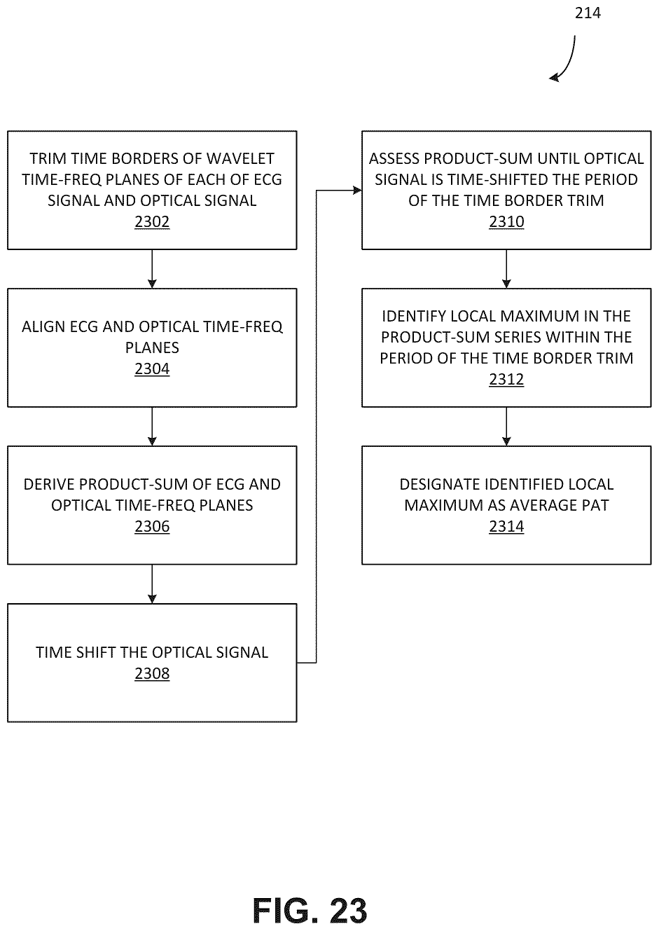

The method 200 also includes calculating PAT based on the converted and normalized R-wave information from the ECG signal and the converted and normalized optical signal (in block 214). In general, the ECG time-frequency plane is used as a basis pattern against which the optical time-frequency plane is assessed for the pattern to determine a time-shift with the greatest product-sum, which can be taken to represent the time delay where the wave systems are best aligned. Referring to FIG. 23, an example of calculating PAT (block 214) is shown. For instance, calculating PAT generally includes trimming the time borders of the wavelet time-frequency planes of each of the ECG and optical signals (in block 2302), aligning the ECG and optical time-frequency planes (in block 2304), deriving the product-sum of ECG and optical time-frequency planes (in block 2306), time shifting the optical signal (in block 2308), assessing the product-sum until the optical signal is time-shifted the period of the time border trim (in block 2310), identifying the local maximum in the product-sum series within the period of the time border trim (in block 2312), and designating the identified local maximum as the average PAT (in block 2314). Each of these steps is described in further detail herein.

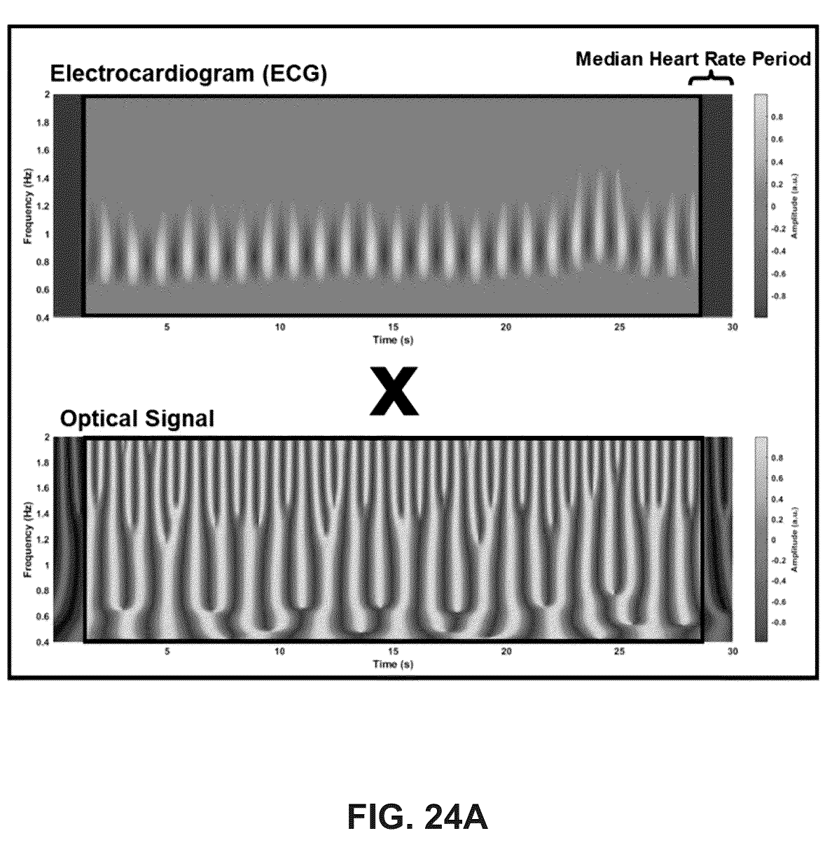

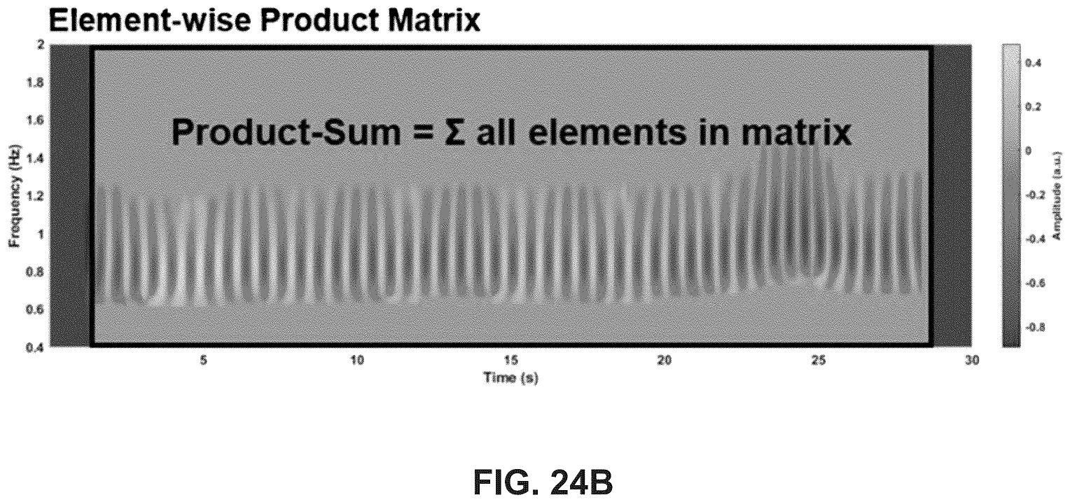

In block 2302, the wavelet time-frequency planes of the ECG and optical signals are trimmed from both time borders (e.g., at the beginning and the end signal) by a time period. In implementations, the time period is the period of the median heart rate as determined by the median of the wavelet derived heart rates. Alternatively, the period could be the average heart rate, however the median heart rate can account for outliers to provide additional reliability in the PAT calculation. Referring to FIG. 24A, each of the example ECG signal wavelet time-frequency plane (e.g., obtained from block 1806) and the example optical signal wavelet time-frequency plane (e.g., obtained from block 212) are shown trimmed at the beginning and the end by a period of the median heart rate.

The method block 214 also includes aligning the ECG and optical time-frequency planes (in block 2304) and deriving the product-sum of ECG and optical time-frequency planes (in block 2306). Referring to FIG. 24A, an example of the alignment of the ECG and optical time-frequency planes is shown with a resultant elementwise product-sum illustrated in FIG. 24B. Following trimming, the remaining elements in the ECG and optical time-frequency planes are element-wise multiplied to form a product matrix, where the elements in the product matrix are summated to provide a final product-sum that is a scalar value. For instance, the sum of the aligned elementwise products can be assessed as an indication of the pattern similarity between the two manifolds, where the ECG time-frequency plane serves as the basis pattern and the optical time-frequency plane is assessed for the pattern. When the optical time-frequency plane is elementwise multiplied by the ECG time-frequency plane, amplitudes outside the heart rate band in the ECG time-frequency plane are reduced to zero, and the frequencies that are likely to be associated with the heart rate are naturally weighted by the large high-probability amplitudes in the ECG time-frequency plane. As such, the product-sums are naturally weighted to contain information about the wave-trains associated with the pulse waves.

The method block 214 includes time shifting the optical signal (in block 2308) and assessing the product-sum until the optical signal is time-shifted the period of the time border trim (in block 2310). For example, the position of the trimmed ECG time-frequency plane (e.g., from block 2302) is maintained, while the optical time-frequency plan is time-shifted in the future. Past shifting may not be utilized, since the pulse wave is a traveling wave and would not appear in the optical signal before its event appears in the ECG signal. For instance, the optimal pattern alignment between the ECG and optical time-frequency planes is expected to occur at later times in the optical signal relative to the ECG signal. The optical time-frequency plan is time-shifted iteratively and the product-sum can be assessed for each time-shift until the optical signal has been time-shifted the period of the median heart rate.

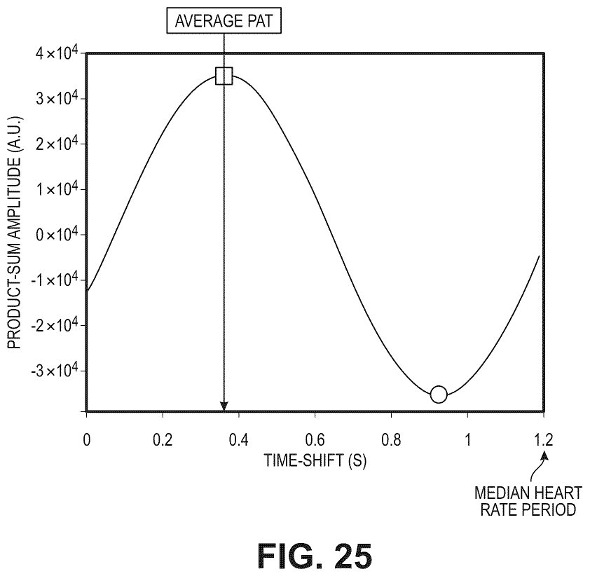

The method block 214 also includes identifying the local maximum in the product-sum series within the period of the time border trim (in block 2312) and designating the identified local maximum as the average PAT (in block 2314). For instance, the ECG and optical signals are reduced to sinusoids so the pattern of the product-sum (e.g., from block 2310) follows a sinusoidal pattern, where the time-shift that expresses a local maximum in the product-sum series within the period of the median heart rate is interpreted as the average PAT of the pulse waves in the sample, as shown for example in FIG. 25.

The method 200 converts time series data to the wavelet time-frequency plane with data normalization, which can provide several advantages over maintaining time-domain techniques that incorporate geometry-based methods (e.g., an intersecting tangents method). For example, conversion of the time series data to the time-frequency plan with the wavelet transform allows for the isolation of information associated with the cardiac rhythm and eliminates information in the series associated with other phenomenon. Additionally, the method 200 mitigates the effects of potential motion artifacts in the sensor data through amplitude normalization, which ensures that the product-sum is not weighted heavily towards high amplitude motion artifacts, which improves the robustness of PAT derived with method 200. Furthermore, method 200 can utilize pattern recognition techniques to derive PAT instead of derivation of individual PATs of time-adjacent pulse waves, which attenuates the influence that outliers may have on the calculation of the average PAT.

The method 200 can also include determining a measurement quality of the determined PAT (in block 216). For example, the method 200 can include a synchronization technique (e.g., synchronization index) to provide feedback about the quality of the derived PAT value. For instance, synchronization can refer to two wave systems of the same frequency sharing the same angular velocity, which means that the phase angle difference between the waveforms will not change across time. Two wave systems that demonstrate synchronization are taken to have a similar origin, whereas two wave systems that do not demonstrate synchronization are taken to be unrelated. Regarding pulse wave trains in the ECG signal and the optical signal, the wave-trains are expected to exhibit synchronization because they originate from the same source (i.e., the heart). If the ECG and optical signals are not exhibiting synchronization, one or more of the following can apply: (1) an error was made in the isolation of the heart rate band in the ECG signal, (2) a motion artifact occurring in the same frequency bandwidth as the heart rate exists in either the ECG signal or the optical signal, or (3) the vascular system is not in steady-state and is changing to a new system state. Since each of these events may have an influence on the derivation of the average PAT value and may also reduce the synchronization of the ECG and optical signals, synchronization can be an appropriate index to measure the quality of the PAT value derived from the methods described herein.

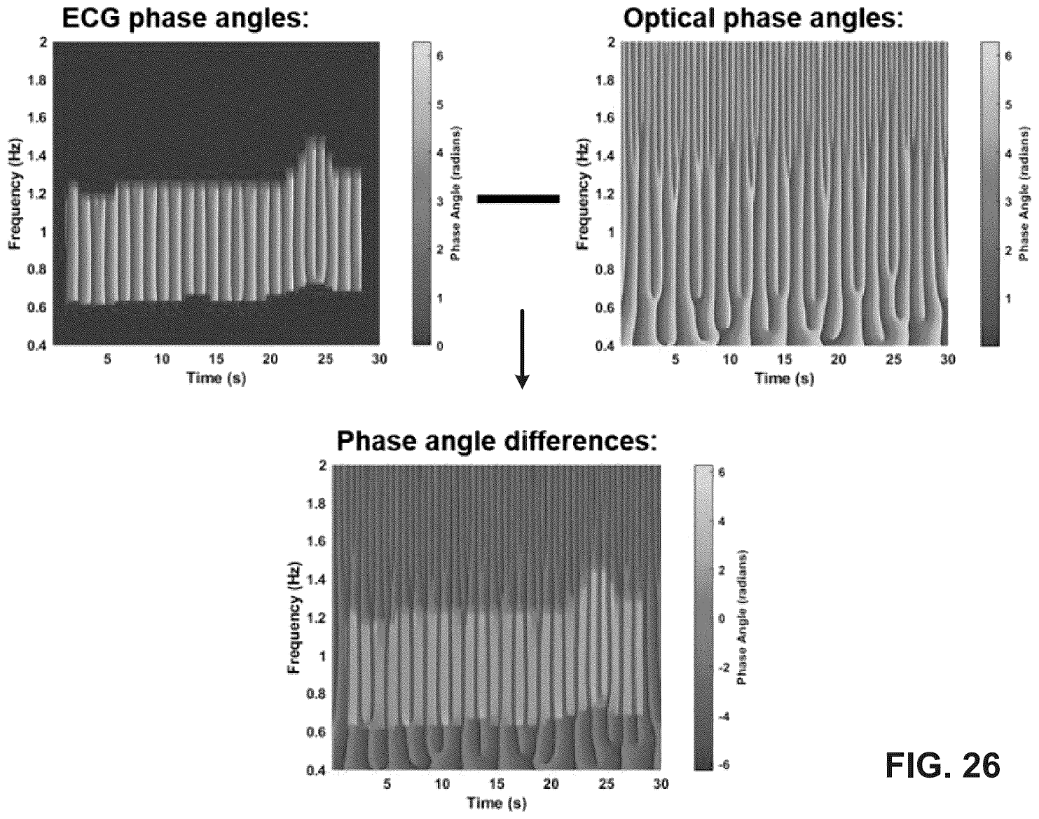

In implementations, phase angles for the derivation of the synchronization index are derived by calculating the phase angles (ranging from 0 to 2π) of the oscillations in the ECG and optical time-frequency planes with their respective wavelet coefficients, followed by calculation of the phase angle differences by elementwise subtraction of the optical signal phase angles from the ECG signal phase angles, as example of which is shown in FIG. 26. Positive phase angle differences indicate that the optical signal wave-train appears after the ECG wave-train and negative phase angle differences indicate that the optical signal wave-train precedes the ECG wave-train.

A general synchronization index between two wave systems can be defined as:

S(t_{i1:i2})=\sqrt{mean (sin ϕ_{i1:i2})^{h2}+mean(cos ϕ_{i1:i2})²} (Eq. 4)

where S(t_{i1:i2}) is the synchronization index for times ‘i1’ through ‘i2’ or ‘index 1’ through ‘index 2,’ and ϕ_{i1:i2} are the phase angle differences from time index ‘i1’ through time index ‘i2.’ The synchronization index is free to vary continuously from ‘0’ to ‘1,’ with ‘0’ indicating no synchronization and ‘1’ indicating perfect synchronization. The method 200 can utilize a modified synchronization index to account for sinus arrythmia, for example by including a weighted average of the phase angle differences between the ECG and optical time-frequency planes. In implementations, the weighting is provided by the cardiac rhythm isolated ECG amplitude time-frequency plane by elementwise multiplication of the amplitude time-frequency plane and the sine and cosine of the phase angle differences time-frequency plane. For example, the modified synchronization index can be defined as:

$$S ( {t}_{i 1 : i 2} ) = \sqrt{\begin{matrix}{( \sum ( ( sin {ϕ}_{i 1 : i 2} ) ( weights ) ) / \sum weights )}^{2} + \\ {( \sum ( ( cos {ϕ}_{i 1 : i 2} ) ( weights ) ) / \sum weights )}^{2}\end{matrix}} \qquad ( Eq . 5 )$$

The modified synchronization index eliminates phase angle differences that are irrelevant to the cardiac related wave-trains and gives preference to phase angle differences that are associated with high probability frequencies in the time-frequency plane, thereby providing an indication as to how well the wave-trains used in the assessment of PAT align. Furthermore, the modified synchronization index may not require the wave systems maintain a constant frequency, which is a constraint of a standard synchronization index, resulting in the modified synchronization index being more reliable for the constantly varying sinus arrythmia.

In implementations, determination of PAT can include receiving data from continuous ECG monitoring (e.g., through use of a chest strap or other device), receiving ECG data intermittently, receiving data on-demand (e.g., using the individual's contralateral hand to initiate data production), or combinations thereof. In implementations, the determined PAT values can be used to assess blood pressure by the principles of the Moens-Korteweg equation.

As described herein, the method 200 can also include determining a measurement quality of the determined PAT (in block 216). For example, the method 200 can include a synchronization technique (e.g., synchronization index) to provide feedback about the quality of the derived PAT value, and in implementations, can be used to determine the PAT value, such as through a multi-resolution synchronization. Synchronization can refer to the phase similarity of two waves as they move through time. When two waves of the same frequency are synchronized, their oscillations are highly correlated. Whereas when two waves of the same frequency are not synchronized, their oscillations are not correlated. When a pair of waves are synchronized, it can indicate that their oscillations may originate from a common source. Whereas, if a pair of waves are not synchronized, it can indicate that their oscillations may not share a common source. Since ECG and optical signals both contain oscillations that originate from a common source (i.e., the heart), synchronization of the oscillations originating from the heart is an invariant characteristic of the ECG and optical signals. Therefore, regions in time where the ECG and optical signals are not well synchronized may not be considered for the calculation of the PAT value, since these regions may be associated with errors.

Based on the synchronization equation (e.g., equation 4 above), it can be appreciated that synchronization between two signals can be calculated across a non-zero time differential, where synchronization is calculated over a window of time rather than a single point in time. The modified synchronization index associated with equation 5 above can be utilized to estimate the quality of a determined PAT value and can thereby be used as a criterion to eliminate erroneous determinations of PAT, however such technique can be limited in its ability to facilitate PAT calculation based on sub-optimal data. A multi-resolution synchronization index can be utilized to estimate the synchronization between ECG and optical data at each finite point in time and thereby assign a quality index to each time point, rather than a single quality index for the whole time series. The multi-resolution synchronization index can attenuate points in time with low synchronization while preserving points in time with high synchronization. Multi-resolution synchronization can therefore bias the calculation of PAT towards time points that demonstrate strong synchronization between ECG and optical signals.

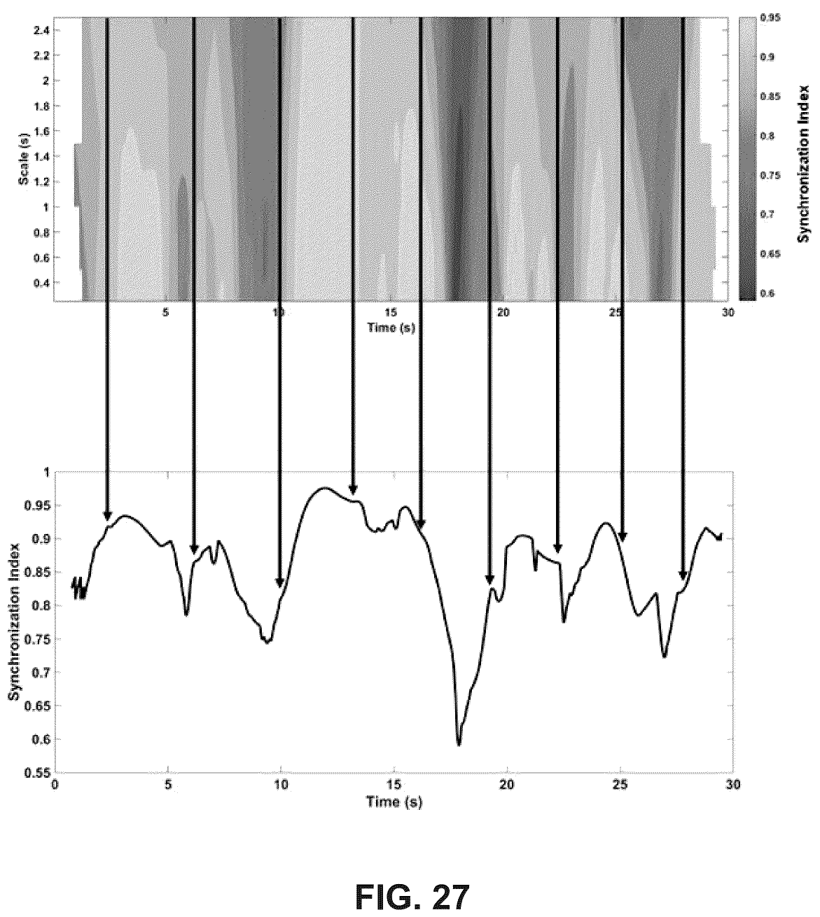

In implementations, block 216 of method 200 includes calculating the synchronization index in sliding windows of different time scales (e.g., similar to a moving average). Reviewing synchronization across different time scales can be beneficial since a small time-window can detect rapid losses of synchronization but may be insensitive to losses of synchronization on larger time scales, whereas a large time-window is sensitive to the loss of synchronization on large time scales, whereas it is insensitive to losses of synchronization on small time scales. In implementations, the method 200 includes multi-resolution synchronization using windows that are scaled between 0.5 and 2.5 seconds to detect synchronization on small and large time scales. While the example includes windows scaled between 0.5 and 2.5 seconds, the disclosure is not limited to such scale windows and can include additional or alternative scale windows dependent on features in the ECG or optical signal. For each scale, a window is generated that is the size of the desired scale (sampling rate (Hz)*scale (s)), and this window is slid across the entire time series with each iteration moving the window one sample. The synchronization indices for each scale are saved in one-dimensional time series that are combined into a matrix of synchronization indices representing the synchronization between the ECG and optical signals as a function of time and scale, an example of which is shown in FIG. 27. To generate a multi-resolution synchronization index value for each point in time, the minimum synchronization index can be taken across all the scales for each point in time, as shown in FIG. 27.

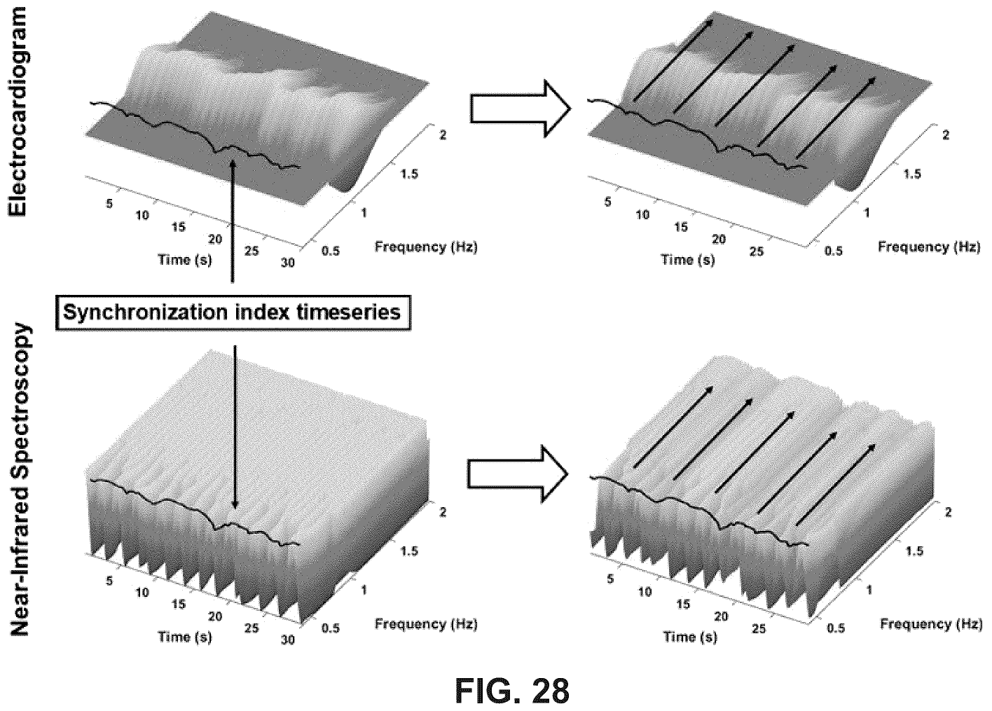

With multi-resolution synchronization, the synchronization index matrix can be reduced to a one-dimensional time series that can be used to threshold the ECG and optical signals before the calculation of PAT, whereas the modified synchronization index was calculated as a “weighted synchronization” as described herein. For multi-resolution synchronization, once a synchronization index has been assigned to each point in time, the one-dimensional time series of synchronization index values can be used to threshold the ECG and optical signals, which in method 200 are converted from one-dimensional time series to a two-dimensional complex time-scale plane. The time dimensions of these planes are the same lengths as the one-dimensional synchronization index vector. Therefore, the synchronization vector can be elementwise multiplied across all frequencies in the time frequency plane, an example of which is shown in FIG. 28. With multi-resolution synchronization, the coefficients at a given time point are all scored with one synchronization index, which represents a quality score for a given point in time. Following the elementwise multiplication of multi-resolution synchronization, regions in time with low synchronization are given a low amplitude, whereas regions in time with high synchronization are given a high amplitude. This can impact the final calculation of PAT because the dot product will be biased towards the regions with high amplitudes.

Generally, any of the functions described herein can be implemented using hardware (e.g., fixed logic circuitry such as integrated circuits), software, firmware, manual processing, or a combination thereof. Thus, the blocks discussed in the above disclosure generally represent hardware (e.g., fixed logic circuitry such as integrated circuits), software, firmware, or a combination thereof. In the instance of a hardware configuration, the various blocks discussed in the above disclosure may be implemented as integrated circuits along with other functionality. Such integrated circuits may include all of the functions of a given block, system, or circuit, or a portion of the functions of the block, system, or circuit. Further, elements of the blocks, systems, or circuits may be implemented across multiple integrated circuits. Such integrated circuits may comprise various integrated circuits, including, but not necessarily limited to: a monolithic integrated circuit, a flip chip integrated circuit, a multichip module integrated circuit, and/or a mixed signal integrated circuit. In the instance of a software implementation, the various blocks discussed in the above disclosure represent executable instructions (e.g., program code) that perform specified tasks when executed on a processor. These executable instructions can be stored in one or more tangible computer readable media. In some such instances, the entire system, block, or circuit may be implemented using its software or firmware equivalent. In other instances, one part of a given system, block, or circuit may be implemented in software or firmware, while other parts are implemented in hardware.

### Example Experimental Determination of Noise Resistance

In an example experiment involving 17 human subjects, near-infrared spectroscopy data and ECG data was collected to assess PAT, correlation coefficients, product-sum, and synchronization index performance for a range of signal-to-noise ratios utilizing the methods described herein (e.g., method 200). The near-infrared spectroscopy data was collected on the medial forearm with a 3.5 cm inter-optode distance, with the ECG data measured synchronously (250 Hz) for 30 minutes for each subject. Ten random starting points from each 30 minute data recording were selected to produce ten 30-second time series for each subject, for a total of 170 time series. White noise was added to the ECG signals to produce 18 signal-to-noise ratios: 0.03125, 0.0625, 0.125, 0.25, 0.5, 1, 2, 4, 8, 16, 32, 64, 128, 256, 512, 1024, 2048, and 4096.

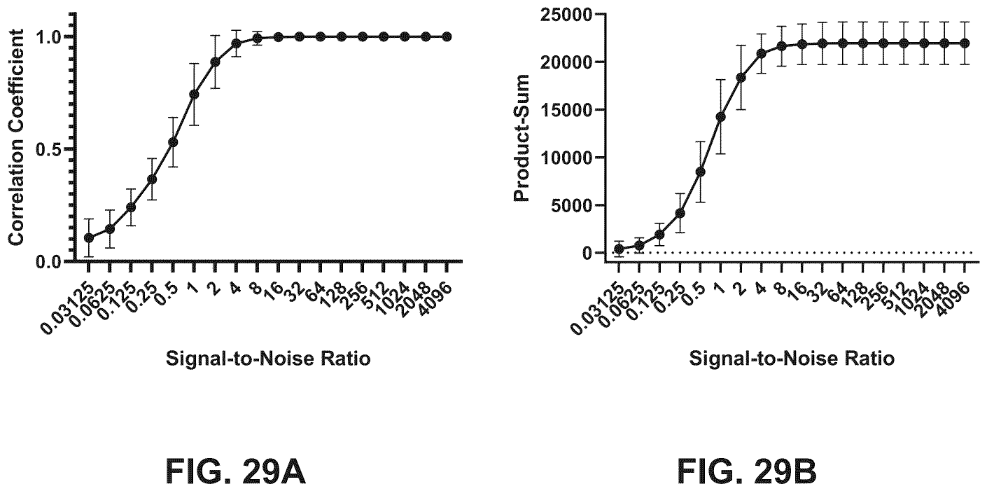

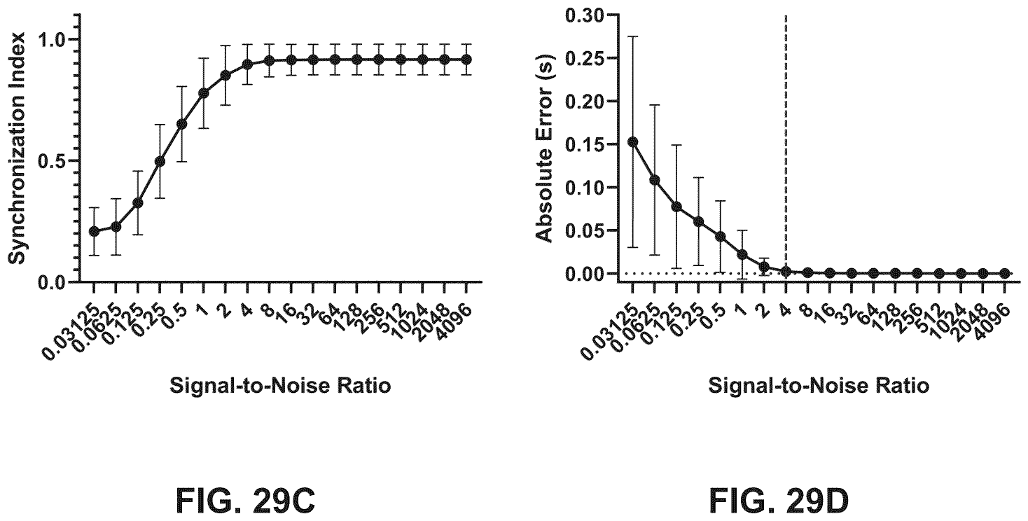

For the correlation coefficient assessment, the matrix of real coefficients from the wavelet transform between 0.4-2 Hz from the unaltered binary ECG signal was compared with the matrices of real coefficients from each noise-injected binary ECG signal, which showed a non-specific indication of similarity between binary ECG signals after noise injection. An example chart of the correlation coefficient for each signal-to-noise ratio set is shown in FIG. 29A.

For the product-sum assessment, the ECG treatment strategy described herein with reference to method 200 was applied to the unaltered and noise-injected ECG signals, and the cardiac-rhythm band was isolated as described with the real components isolated. The product-sum was calculated between each noise-injected coefficient matrix and the unaltered binary ECG coefficient matrix, which showed a specific indication of the similarity between binary ECG signals after noise injection (e.g., associated with the cardiac-rhythm). An example chart of the product-sum for each signal-to-noise ratio set is shown in FIG. 29B.

For the PAT and synchronization index assessment, the synchronization index was calculated for each iteration for comparison with the correlation coefficient and the product-sum. An example chart of the synchronization index for each signal-to-noise ratio set is shown in FIG. 29C. The PAT was calculated for each iteration and the absolute error was calculated with respect to the no-noise condition. The absolute PAT error was compared with the synchronization index at each signal-to-noise ratio, an example chart of which is shown in FIG. 29D. The synchronization index was shown to diverge with the absolute PAT error determined, providing a good indication of signal quality.

### Example Experimental Determination of PAT

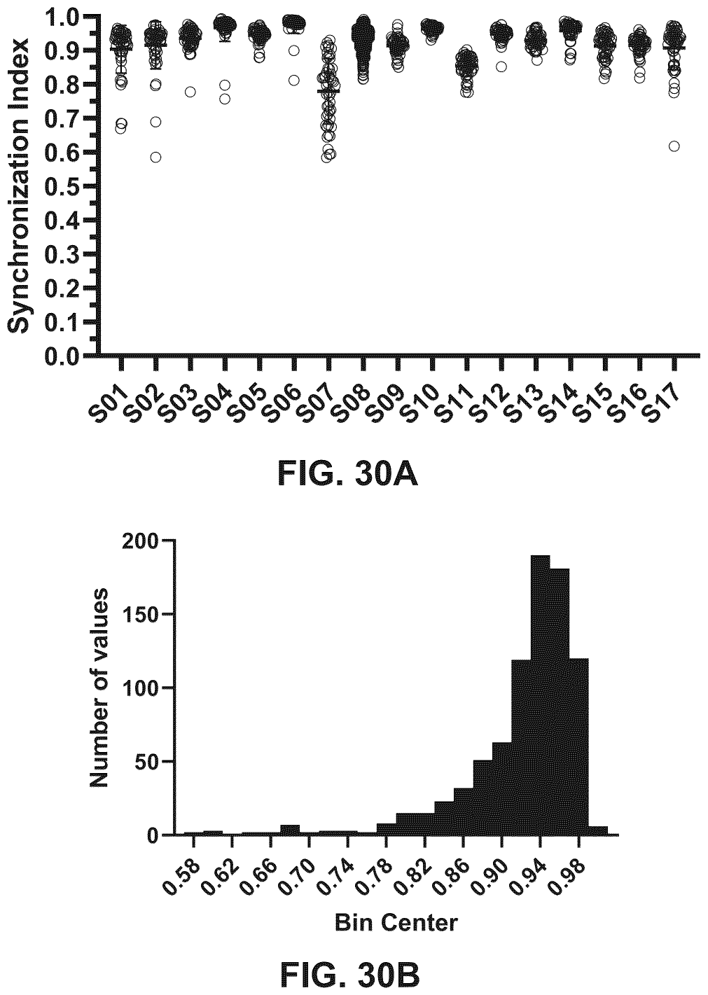

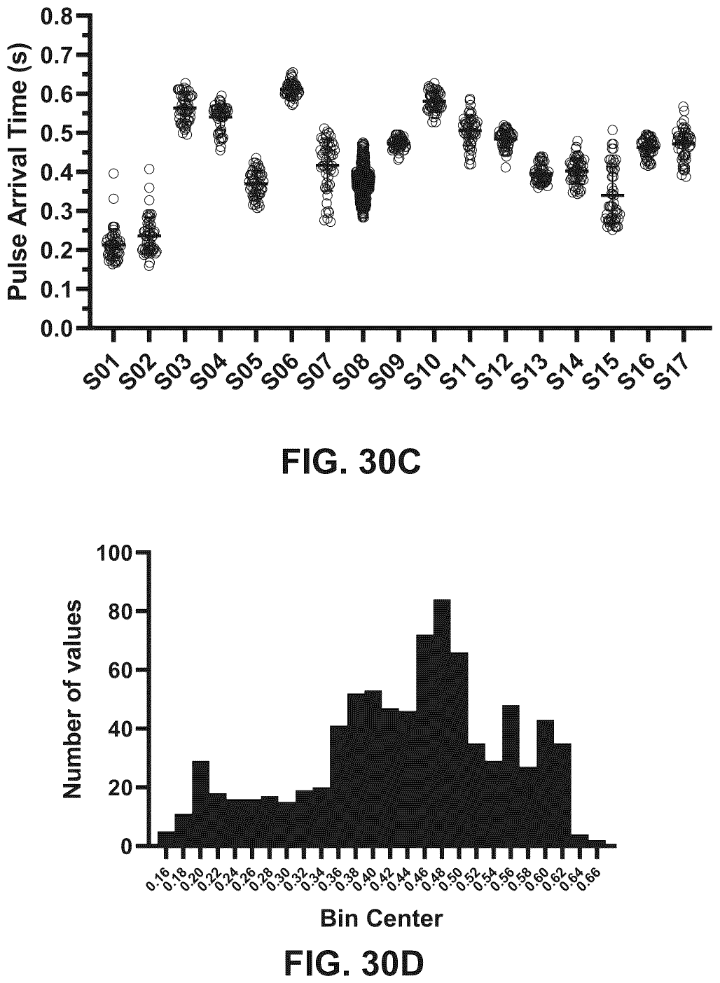

In an example experiment involving 17 human subjects, near-infrared spectroscopy data and ECG data was collected to assess PAT and synchronization index performance for each iteration utilizing the methods described herein (e.g., method 200). The near-infrared spectroscopy data was collected on the medial forearm with a 3.5 cm inter-optode distance, with the ECG data measured synchronously (250 Hz) for 30 minutes for each subject. Fifty random starting points from each 30 minute data recording were selected to produce fifty 30-second time series for each subject, for a total of 850 time series. An example chart of the synchronization index for each subject is shown in FIG. 30A and an example chart of the number of values for data points for synchronization is shown in FIG. 30B. The synchronization index resulted in a generally high index (e.g., around 9) and followed a negative skewed distribution. An example chart of the PAT determined for each subject is shown in FIG. 30C and an example chart of the number of values for data points for PAT is shown in FIG. 30D. The determined PAT values demonstrated appropriate variability between and within the individual subjects.

### Example Experimental Multi-Resolution Synchronization

An example experiment involving 17 human subjects was performed to test the efficacy of multi-resolution synchronization to modify PAT calculation quality utilizing the multi-resolution synchronization methods described herein (e.g., method 200). Each subject was instrumented with a NIRS sensor on the medial forearm and a three-lead ECG. The subjects rested quietly in the supine position for 30 min while NIRS and ECG were recorded continuously. From each 30 min time series for each subject, 10 randomly selected 30 sec intervals were selected for further analysis for a total of 170 recordings. Simulations were then generated for each recording to introduce artificial errors into the ECG and NIRS signals. The errors were made to simulate errors that may occur during signal acquisition. For example, data were either erased or added to the NIRS signal to simulate failure of data acquisition or motion artifacts, respectively. Furthermore, R-waves were either added in places that were incorrect or R-waves that existed were eliminated. Each 30 sec recording was then subjected to 11 simulations for a total of 1870 simulations. The first simulation had no artificial errors, and each subsequent simulation introduced 2 random errors, up to a total of 20 errors. For each simulation, PAT and the synchronization index were calculated over the whole 30 sec recording. The described procedure was performed once without the use of multi-resolution synchronization and once with the use of multi-resolution synchronization for a total of 3740 simulations. The randomly generated simulations used without multi-resolution synchronization were saved and used for testing multi-resolution synchronization.

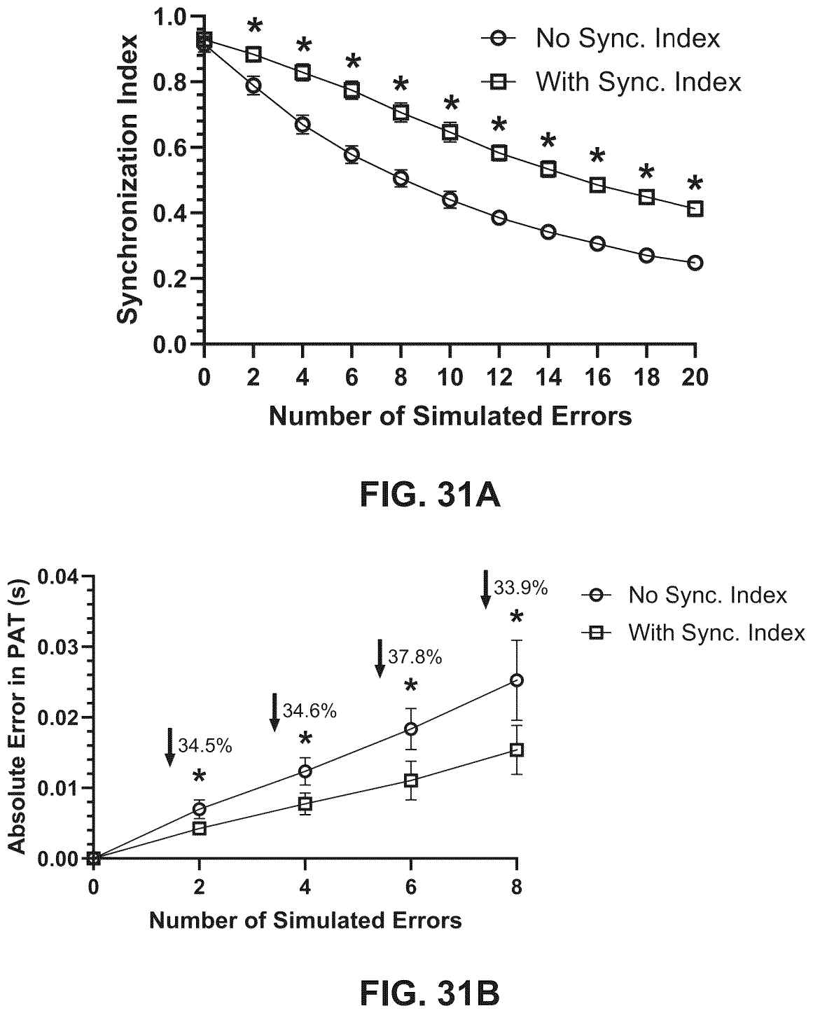

Results of the analysis indicated that as the number of errors in the 30 sec recordings increased, the synchronization between NIRS and ECG decreased, as shown in FIG. 31A. Furthermore, according to a 2-way analysis of variance, it was determined that when multi-resolution synchronization was used to attenuate regions of low synchronization, the synchronization of the 30 sec recordings was significantly higher compared to when multi-resolution synchronization was not applied, as shown in FIG. 31A. Additionally, as shown in FIG. 31B, it was determined that as the number of errors increased, the error in the PAT calculation increased (e.g., absolute error relative to simulation with no artificial errors). For instance, according to a 2-way analysis of variance, it was determined that when multi-resolution synchronization was applied, the error in the PAT calculation was significantly reduced up to 8 simulated errors (with an approximately 35% reduction in error). There were no statistically determined benefits for the multi-resolution synchronization beyond 8 simulated errors in the experiment.

### Example Experimental Determination of Blood Pressure Change Detection

An example experiment involving 44 human subjects was performed to test the efficacy of PAT determination in response to changes in blood pressure using the methods described herein (e.g., method 200). According to the Bramwell-Hill equation (related to Moens-Kortweg relationship), the time it takes for a pressure wave to propagate through an elastic pipe is proportional to the pressure differential across the pipe system. Therefore, since pulse arrival time (PAT) is an index of the propagation time of the pulse pressure wave to traverse from the heart to the microvasculature, PAT may be associated with changes in blood pressure. Since PAT can be derived from near-infrared spectroscopy (NIRS) and electrocardiogram (ECG) traces (e.g., as described through method 200 herein), which are available in current smartwatch technologies, PAT may offer a method to detect changes in blood pressure for smartwatch users. The methods described herein facilitate calculating PAT that makes it resilient against signal acquisition errors (e.g., multi-resolution synchronization), which may make it a more accurate algorithm to detect blood pressure changes in smartwatch users.

During the experiment, the subjects were reclined in the supine position for 20-min before signal acquisition. Synchronous measurements were taken with NIRS on the right medial forearm, three-lead ECG, and arterial blood pressure with Finapres (Ohmeda, Madison, U.S.A.) from the right middle finger. To increase blood pressure, subjects isometrically squeezed a handgrip dynamometer with their left hand at 50% of their maximal grip strength for 1-min, while blood flow was occluded to the exercising forearm with a rapid inflation cuff (250 mmHg). For each subject, PAT was calculated with NIRS and ECG over ten overlapping 30-sec windows within each 1-min recording. The average systolic, diastolic, and mean blood pressures from Finapres were also calculated across each 30-sec window. For each subject, statistical models relating PAT to systolic, diastolic, and mean blood pressures were derived from the ten sampled points during isometric handgrip exercise. It was determined that a quadratic function was appropriate to represent the relationship between PAT and blood pressure. The variance in PAT that the models explained (i.e., coefficient of determination) was used to determine the sensitivity of the methods described herein to blood pressure. If the coefficient of determination was less than 0.5, this was considered a weak relationship and was considered a poor detection of the relationship between PAT and blood pressure. If the coefficient of determination was greater than 0.5, this was considered a successful detection of the relationship between PAT and blood pressure. Specifically, if the coefficient of determination was between 0.5 and 0.8, this was considered a strong relationship between PAT and blood pressure. And, if the coefficient of determination was greater than 0.8, this was considered a very strong relationship between PAT and blood pressure.

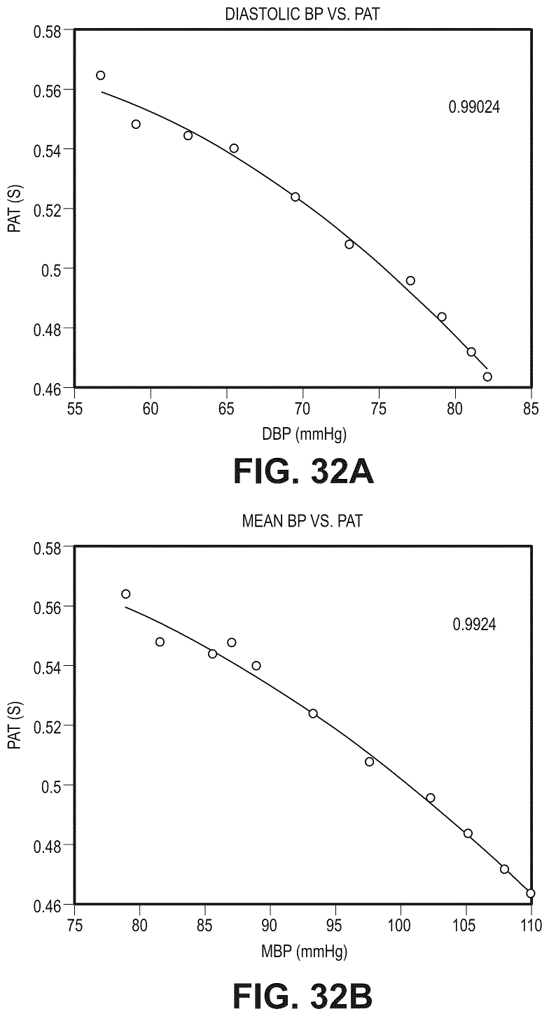

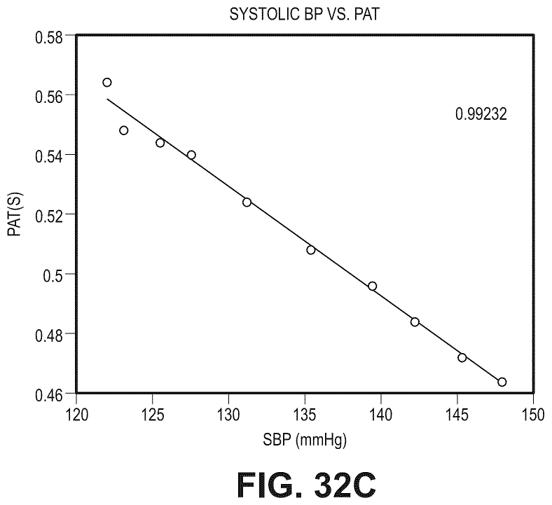

Diastolic, systolic, and mean blood pressures increased during the 1-min of isometric handgrip exercise for the subjects. The methods described herein successfully captured the relationship between diastolic blood pressure and PAT in 38 out of 44 subjects (86.37%) in the experimental conditions, whereas 6 of the subjects had a weak correlation (r²<0.5). Furthermore, of the 38 subjects that successfully captured the relationship between diastolic blood pressure and PAT, 3 models were considered strong (0.5<r²<0.8), and 35 models were considered very strong (r²>0.8). The methods described herein successfully captured the relationship between mean blood pressure and PAT in 38 out of 44 subjects (86.37%), whereas 6 of the subjects had a weak correlation (r²<0.5). Furthermore, of the 38 subjects that successfully captured the relationship between mean blood pressure and PAT, 6 models were considered strong (0.5<r²<0.8), and 32 models were considered very strong (r²>0.8). The methods described herein successfully captured the relationship between systolic blood pressure and PAT in 34 out of 44 subjects (77.28%), whereas 10 of the subjects had a weak correlation (r²<0.5). Furthermore, of the 34 subjects that successfully captured the relationship between systolic blood pressure and PAT, 10 models were considered strong (0.5<r²<0.8), and 24 models were considered very strong (r²>0.8). Examples from one subject of models between PAT and blood pressure derived with the methods described herein are shown in FIGS. 32A through 32C, where FIG. 32A illustrates experimental results of the relationship between PAT and diastolic blood pressure from one test subject using multi-resolution synchronization, FIG. 32B illustrates experimental results of the relationship between PAT and mean blood pressure from one test subject using multi-resolution synchronization, and FIG. 32C illustrates experimental results of the relationship between PAT and systolic blood pressure from one test subject using multi-resolution synchronization.

### Conclusion

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

1. A mobile electronic device, comprising:
a sensor configured to provide electrocardiogram (ECG) data from a user;
an optical sensor configured to provide optical data from the user; and
a controller configured to access each of the ECG data and the optical data, the controller configured to:
isolate and normalize R-wave information from the ECG data,
isolate information associated with the cardiac rhythm from the isolated and normalized R-wave information to provide pulse waves,
determine temporal characteristics of the pulse waves,
convert and normalize the optical data in a wavelet time-frequency plane, and
calculate pulse arrival time utilizing each of the temporal characteristics of the pulse waves and the converted and normalized optical data.

2. The mobile electronic device of claim 1, wherein the controller is further configured to determine a measurement quality of the calculated pulse arrival time.

3. The mobile electronic device of claim 2, wherein the controller is configured to utilize a synchronization index to determine the measurement quality of the calculated pulse arrival time, wherein the synchronization index eliminates phase angle differences that are irrelevant to the cardiac related wave-trains and gives preference to phase angle differences that are associated with high probability frequencies in the time-frequency plane.

4. The mobile electronic device of claim 1, wherein the controller is further configured to utilize a multi-resolution synchronization index to calculate pulse arrival time.

5. The mobile electronic device of claim 4, wherein the multi-resolution synchronization index attenuates points in time with low synchronization between the optical data and the ECG data while preserving points in time with high synchronization between the optical data and the ECG data.

6. The mobile electronic device of claim 1, wherein the controller is configured to isolate and normalize R-wave information from the ECG data through:
filtering the ECG data to provide filtered ECG data;
replacing negative values from the filtered ECG data with zero values;
amplifying the positive filtered ECG data values;
removing information not attributed to R-waves from the positive filtered ECG data to provide an amplified ECG signal; and
reducing the amplified ECG signal to a one-dimensional data-set.

7. The mobile electronic device of claim 6, wherein the controller is configured to reduce the amplified ECG signal to a one-dimensional data-set through application of a wavelet transform.

8. The mobile electronic device of claim 6, wherein the controller is further configured to:
determine local maximum values in the one-dimensional data-set, the local maximum values corresponding to locations of R-waves;
create a binary ECG vector containing temporal locations of the R-waves; and
convert the binary ECG vector to a wavelet time-frequency plane.

9. The mobile electronic device of claim 1, wherein the controller is configured to isolate information associated with the cardiac rhythm from the isolated and normalized R-wave information to provide pulse waves through:
creating a one-dimensional time series of heart rates associated with the ECG signal;
superimposing the one-dimensional time series of heart rates onto a wavelet time-frequency plane;
recording a plurality of maximum wavelength amplitudes of the wavelet time-frequency plane within a confidence interval at each point of the one-dimensional time series of heart rates;
isolating amplitudes within the confidence interval from the plurality of maximum wavelength amplitudes; and
normalizing the isolated amplitudes.

10. The mobile electronic device of claim 1, wherein the controller is configured to determine temporal characteristics of the pulse waves through:
creating a wavelet time-frequency plane using real components of wavelet coefficients of a binary ECG vector; and
isolating a portion of the real component wavelet time-frequency plane related to cardiac rhythm.

11. The mobile electronic device of claim 1, wherein the controller is configured to calculate pulse arrival time utilizing each of the temporal characteristics of the pulse waves and the converted and normalized optical data through:
trimming time borders of wavelet time-frequency planes of each of the ECG signal and the optical signal by a trim time period;
aligning the trimmed ECG time-frequency plane and the trimmed optical time-frequency plane;
deriving a product-sum of the trimmed ECG time-frequency plane and the trimmed optical time-frequency plane;
time shifting the trimmed optical time-frequency plane relative to the trimmed ECG time-frequency plane;
deriving a product-sum series of the trimmed optical time-frequency plane and the trimmed ECG time-frequency plane until the trimmed optical signal is time-shifted by the trim time period;
identifying a local maximum in the product-sum series within the period of the trim time period; and
designating the identified local maximum as an average pulse arrival time.

12. A method for determining of pulse arrival time, comprising:
receiving electrocardiogram (ECG) data associated with a user from a first sensor;
isolating and normalizing R-wave information from the ECG data;
isolating information associated with the cardiac rhythm from the isolated and normalized R-wave information to provide pulse waves;
determining temporal characteristics of the pulse waves;
receiving optical sensor data associated with the user from a second sensor;
converting and normalizing the optical sensor data in a wavelet time-frequency plane, and
calculating pulse arrival time utilizing each of the temporal characteristics of the pulse waves and the converted and normalized optical sensor data.

13. The method of claim 12, wherein calculating pulse arrival time utilizing each of the temporal characteristics of the pulse waves and the converted and normalized optical sensor data further includes utilizing a multi-resolution synchronization index to calculate pulse arrival time.

14. The method of claim 13, wherein the multi-resolution synchronization index attenuates points in time with low synchronization between the optical data and the ECG data while preserving points in time with high synchronization between the optical data and the ECG data.

15. The method of claim 12, wherein isolating and normalizing R-wave information from the ECG data includes:
filtering the ECG data to provide filtered ECG data;
replacing negative values from the filtered ECG data with zero values;
amplifying the positive filtered ECG data values;
removing information not attributed to R-waves from the positive filtered ECG data to provide an amplified ECG signal; and
reducing the amplified ECG signal to a one-dimensional data-set.

16. The method of claim 15, wherein reducing the amplified ECG signal to a one-dimensional data-set includes applying a wavelet transform to the amplified ECG signal.

17. The method of claim 15, wherein isolating and normalizing R-wave information from the ECG data further includes:
determining local maximum values in the one-dimensional data-set, the local maximum values corresponding to locations of R-waves;
creating a binary ECG vector containing temporal locations of the R-waves; and
converting the binary ECG vector to a wavelet time-frequency plane.

18. The method of claim 12, wherein isolating information associated with the cardiac rhythm from the isolated and normalized R-wave information to provide pulse waves includes:
creating a one-dimensional time series of heart rates associated with the ECG signal;
superimposing the one-dimensional time series of heart rates onto a wavelet time-frequency plane;
recording a plurality of maximum wavelength amplitudes of the wavelet time-frequency plane within a confidence interval at each point of the one-dimensional time series of heart rates;
isolating amplitudes within the confidence interval from the plurality of maximum wavelength amplitudes; and
normalizing the isolated amplitudes.

19. The method of claim 12, wherein determining temporal characteristics of the pulse waves includes:
creating a wavelet time-frequency plane using real components of wavelet coefficients of a binary ECG vector; and
isolating a portion of the real component wavelet time-frequency plane related to cardiac rhythm.

20. The method of claim 12, wherein the calculating pulse arrival time utilizing each of the temporal characteristics of the pulse waves and the converted and normalized optical sensor data includes:
trimming time borders of wavelet time-frequency planes of each of the ECG signal and the optical signal by a trim time period;
aligning the trimmed ECG time-frequency plane and the trimmed optical time-frequency plane;
deriving a product-sum of the trimmed ECG time-frequency plane and the trimmed optical time-frequency plane;
time shifting the trimmed optical time-frequency plane relative to the trimmed ECG time-frequency plane;
deriving a product-sum series of the trimmed optical time-frequency plane and the trimmed ECG time-frequency plane until the trimmed optical signal is time-shifted by the trim time period;
identifying a local maximum in the product-sum series within the period of the trim time period; and
designating the identified local maximum as an average pulse arrival time.
